(19) United States
(12) Patent Application Publication
(10) Pub. No.: US 2025/0144349 A1
(43) Pub. Date: May 8, 2025

(54) POSITIONING AND STABILISING STRUCTURE FOR A PATIENT INTERFACE

(71) Applicants: ResMed Pty Ltd, Bella Vista (AU); ResMed Asia Pte. Ltd., Singapore (SG)

(72) Inventors: Matthew EVES, Sydney (AU); Hugh Francis Stewart THOMAS, Sydney (AU); Maximilian Aji WIJOYOSENO, Singapore (SG)

(21) Appl. No.: 18/937,219

(22) Filed: Nov. 5, 2024

(30) Foreign Application Priority Data
Nov. 6, 2023 (AU) 2023903551

(51) Int. Cl. A61M 16/06

(52) U.S. Cl. CPC A61M 16/0672; A61M 16/0627; A61M 16/0694

(57) ABSTRACT

A positioning and stabilising structure for a patient interface has a pair of side straps, a top strap and a rear strap, the positioning and stabilising structure further including at least one strap with a chin engaging portion configured to bias a patient's chin towards a closed mouth position, to thereby reduce or eliminate mouth leak and/or a mouth blocking member or portion configured to engage and seal against the patient's mouth, to thereby reduce or eliminate mouth leak.

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

### 1 CROSS-REFERENCE TO RELATED APPLICATION

This application claims the benefit of priority to Australian Provisional Application No. 2023903551, filed Nov. 6, 2023, which is incorporated herein by reference in its entirety.

### 2 BACKGROUND OF THE TECHNOLOGY

### 2.1 Field of the Technology

The present technology relates to one or more of the screening, diagnosis, monitoring, treatment, prevention and amelioration of respiratory-related disorders. The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

Certain masks may cause some patients a feeling of claustrophobia, unease and/or may feel overly obtrusive.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

Consequently, some masks suffer from being obtrusive, aesthetically undesirable, costly, poorly fitting, difficult to use, and/or uncomfortable especially when worn for long or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy, especially if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications: WO 1998/004310; WO 2006/074513; WO 2010/135785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Inc. has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications describe examples of nasal pillows masks: International Patent Application WO 2004/073778 (describing amongst other things aspects of the SWIFT™ nasal pillows mask), US Patent Application 2009/0044808 (describing amongst other things aspects of the SWIFT™ LT nasal pillows mask); International Patent Applications WO 2005/063328 and WO 2006/130903 (describing amongst other things aspects of the MIRAGE LIBERTY™ full-face mask); International Patent Application WO 2009/052560 (describing amongst other things aspects of the SWIFT™ FX nasal pillows mask).

### 2.2.3.1.2 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

One technique is the use of adhesives, e.g. see US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 2.2.3.1.3 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

### 2.2.3.1.4 Pressurised Air Conduit Used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

### 2.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

2.2.3.6

### 2.2.3.7 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 2.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiography (ECG), electrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold the seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises patient interface comprising a plenum chamber pressurisable to a therapeutic pressure of at least 4 cmH2O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has a hole therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface also comprises a positioning and stabilising structure to provide a force to hold the seal-forming structure in a therapeutically effective position on the patient's head.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

One form of the present technology comprises a positioning and stabilising structure for a patient interface, the positioning and stabilising structure comprising a pair of side straps, a top strap and a rear strap, the positioning and stabilising structure further comprising an inferior strap, wherein the inferior strap comprises a mouth blocking member or portion configured to engage and seal against the patient's mouth, to thereby reduce or eliminate mouth leak.

The positioning and stabilising structure may include one or more of the following: wherein the rear portion is configured to engage a posterior portion of the patient's head in use and is configured to overlie the occipital bone of the patient's head in use, or lie proximate a junction between the occipital bone and the parietal bones; wherein the top portion is configured to engage a superior portion of the patient's head in use and is configured to overlie the parietal bones of the patient's head in use; wherein the mouth blocking member or portion comprises a mouth engaging portion. made from silicone; wherein a patient facing surface of the silicone is “tacky” to assist in ensuring the lips do not move apart; wherein the mouth blocking member or portion comprises a mouth engaging portion made from a bio-compatible and substantially non-porous material; wherein the mouth engaging portion comprises a textile with a substantially non-porous coating; wherein the mouth engaging portion is formed integrally with the inferior strap; wherein the mouth engaging portion is inset into the inferior strap, or may be connected to a patient facing surface of the inferior strap or portion; and/or wherein the inferior strap is configured to extend inferior to the patient's ears and is connected to the rear strap at the rear of the patient's head.

Another form of the present technology comprises a positioning and stabilising structure for a patient interface, the positioning and stabilising structure comprising a pair of side straps, a top strap and a rear strap, the positioning and stabilising structure further comprising a chin strap comprising a chin engaging portion, the chinstrap configured to bias a patient's chin towards a closed mouth position, to thereby reduce or eliminate mouth leak.

In examples, the positioning and stabilising structure may include one or more of the following: wherein the rear portion is configured to engage a posterior portion of the patient's head in use and is configured to overlie the occipital bone of the patient's head in use, or lie proximate a junction between the occipital bone and the parietal bones; wherein the top portion is configured to engage a superior portion of the patient's head in use and is configured to overlie the parietal bones of the patient's head in use; wherein the chin strap is connected to the side straps, and is configured to be positioned substantially level with and anterior of the patient's ears; wherein the chin engaging portion is wider the than a remainder of the chin strap; wherein the chin engaging portion is shaped to have a substantially concave portion to conform to the patient's chin; wherein, in use, the chin strap is configured to apply a force to the patient's chin which biases the patient's chin towards a position in which the patient's mouth is closed; and/or wherein the chin strap is length adjustable and/or elastic.

According to another aspect of the present technology, there is provided a positioning and stabilising structure for a patient interface, the positioning and stabilising structure comprising a pair of side straps, a top strap and a rear strap, the positioning and stabilising structure further comprising at least one strap comprising a chin engaging portion configured to bias a patient's chin towards a closed mouth position, to thereby reduce or eliminate mouth leak and/or a mouth blocking member or portion configured to engage and seal against the patient's mouth, to thereby reduce or eliminate mouth leak.

In examples, the positioning and stabilising structure may include one or more of the following features: wherein the rear portion is configured to engage a posterior portion of the patient's head in use and is configured to overlie the occipital bone of the patient's head in use, or lie proximate a junction between the occipital bone and the parietal bones, and the top portion is configured to engage a superior portion of the patient's head in use and is configured to overlie the parietal bones of the patient's head in use; wherein the strap is configured to move between a mouth sealing position and a chin supporting position; wherein the strap remains attached to the side straps or the rear strap at the same point of connection, whether in a mouth sealing mode or a chin strap mode, wherein the strap is configured to both seal the mouth and serve as a chin support; wherein the strap is attached to the side straps, the rear strap, or simultaneously to both the side and rear straps.

In examples there may be provided a patient interface for treating a patient with a respiratory disorder, comprising a seal forming portion configured to form a seal with the patient's nasal airways, the seal being configured to form a seal in use with at least the patient's lip superior, nasal alae and a pronasale portion of the patient's nose; and the positioning and stabilising structure as described above, to support the seal-forming portion.

Another aspect of one form of the present technology is a patient interface that is moulded or otherwise constructed with a perimeter shape which is complementary to that of an intended wearer.

An aspect of one form of the present technology is a method of manufacturing apparatus.

Another aspect of one form of the present technology is a method of assembling a modular system comprising selecting a positioning and stabilising structure, and connecting the positioning and stabilising structure to either a first cushion or a second cushion.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

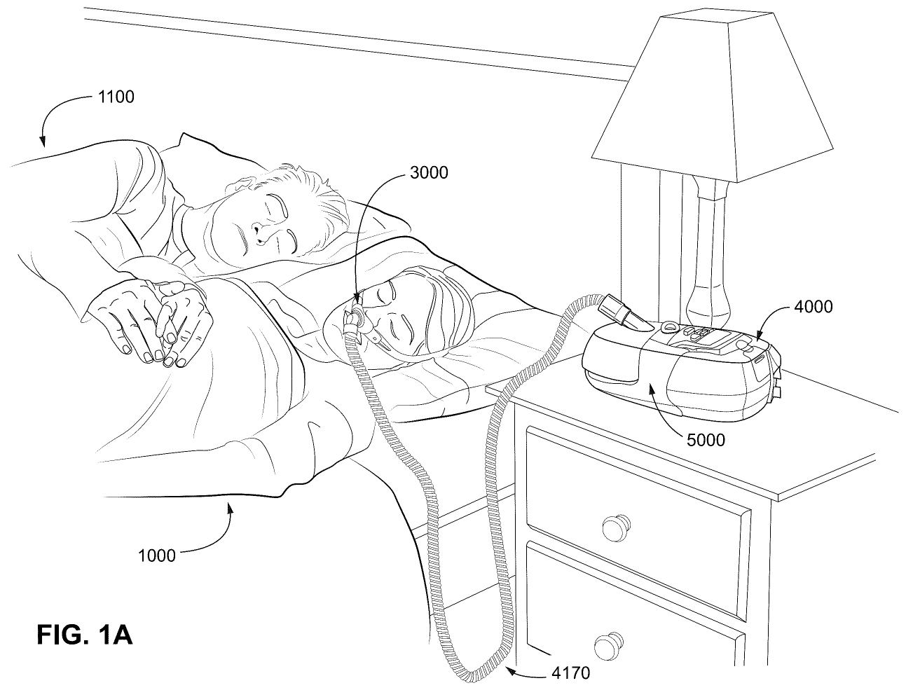

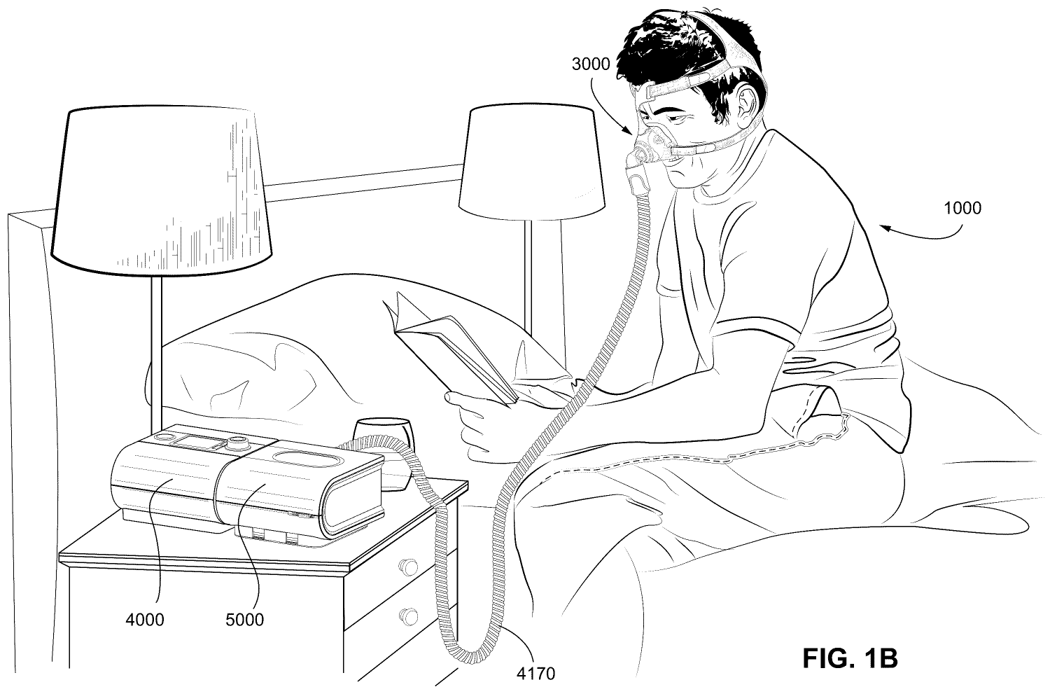

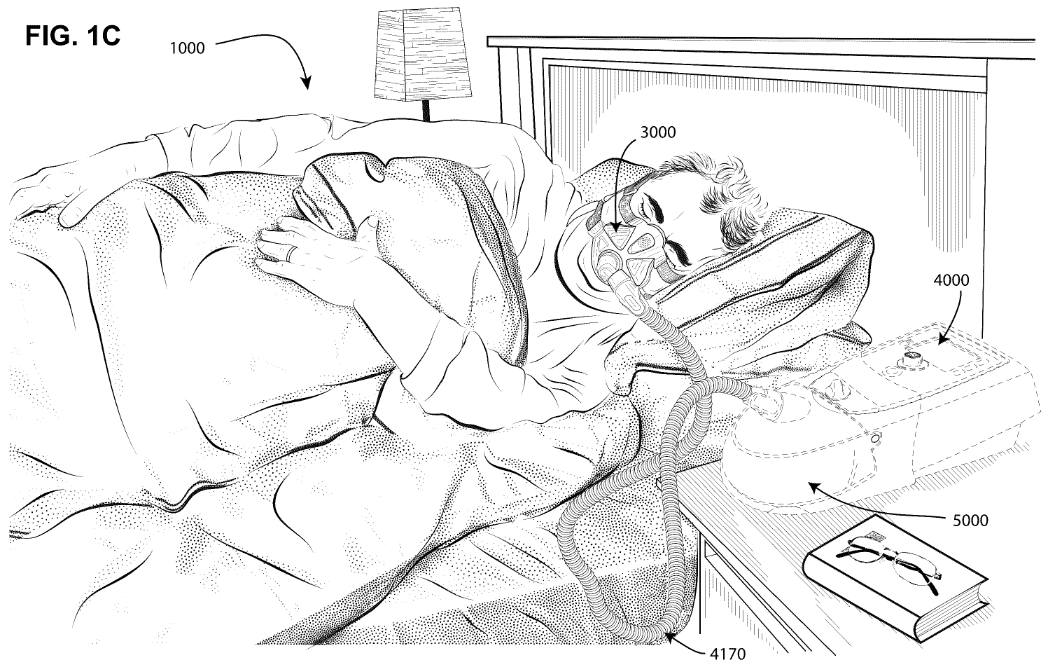

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

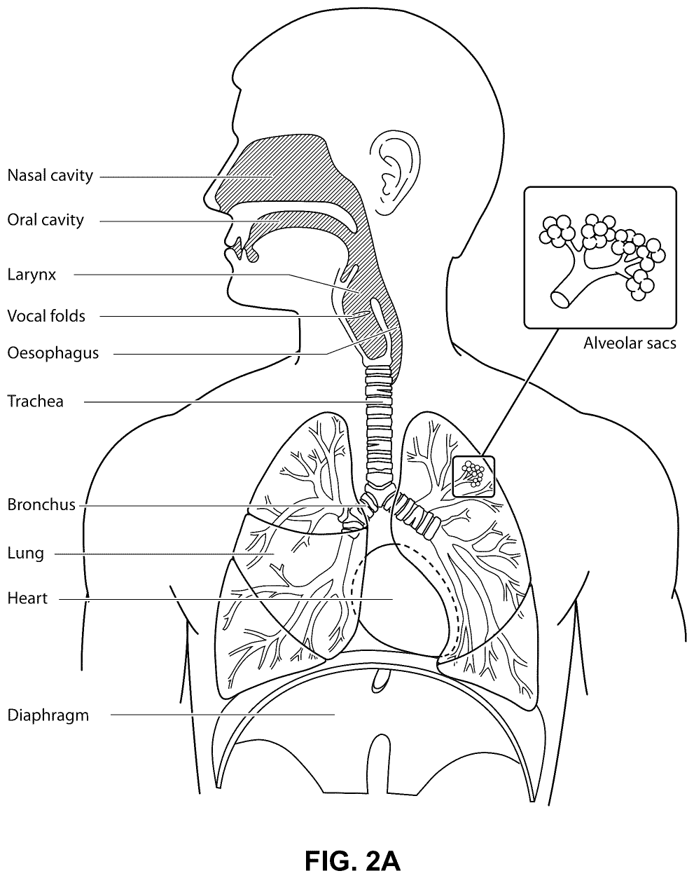

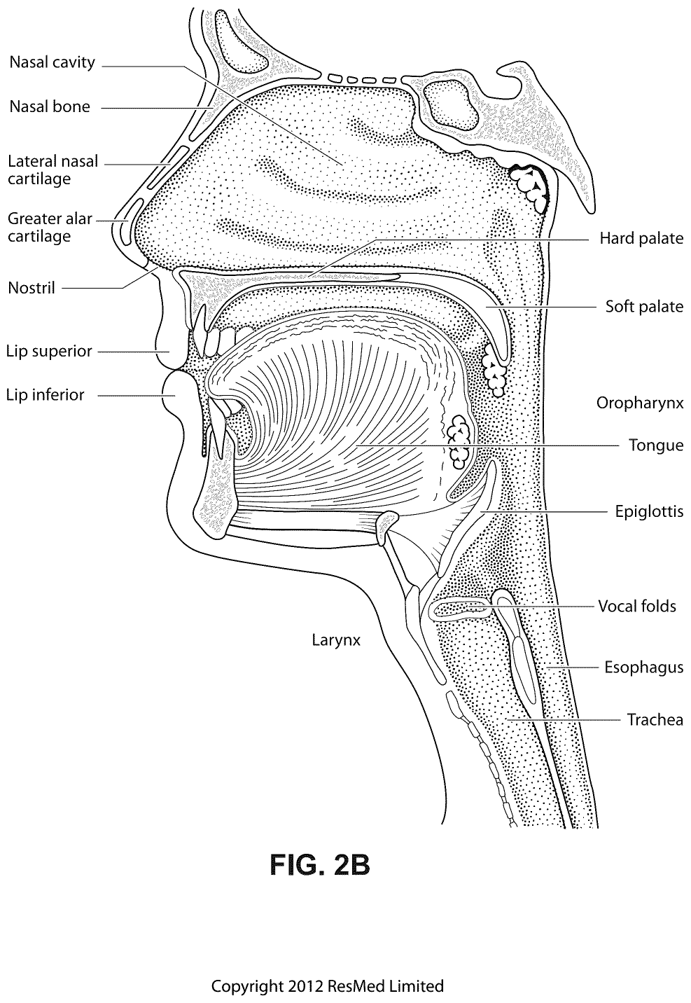

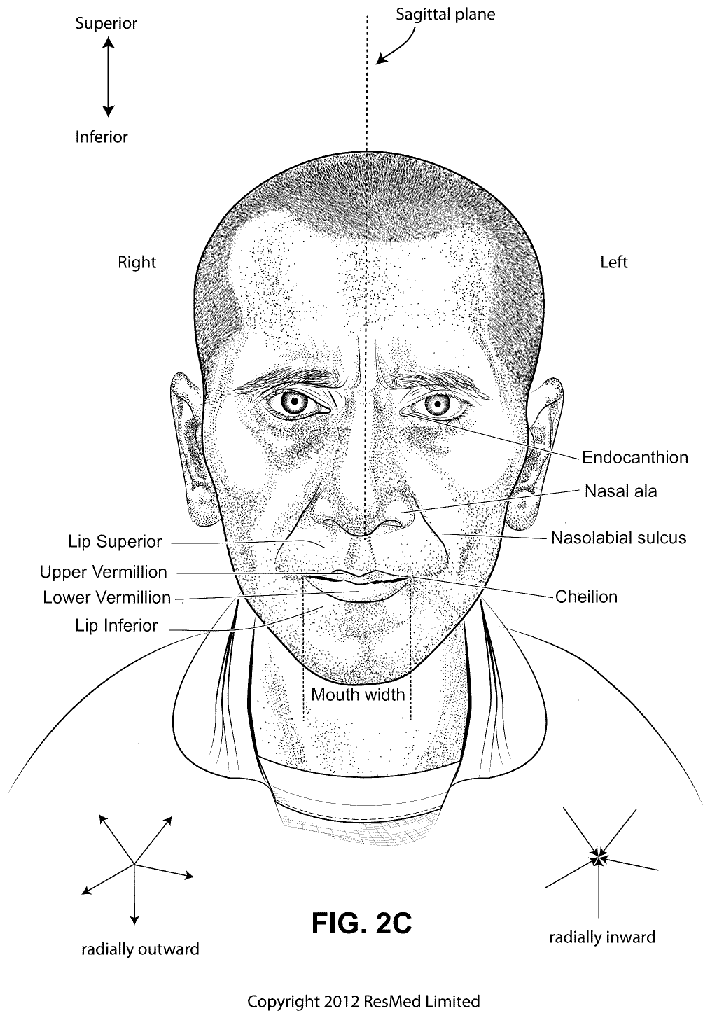

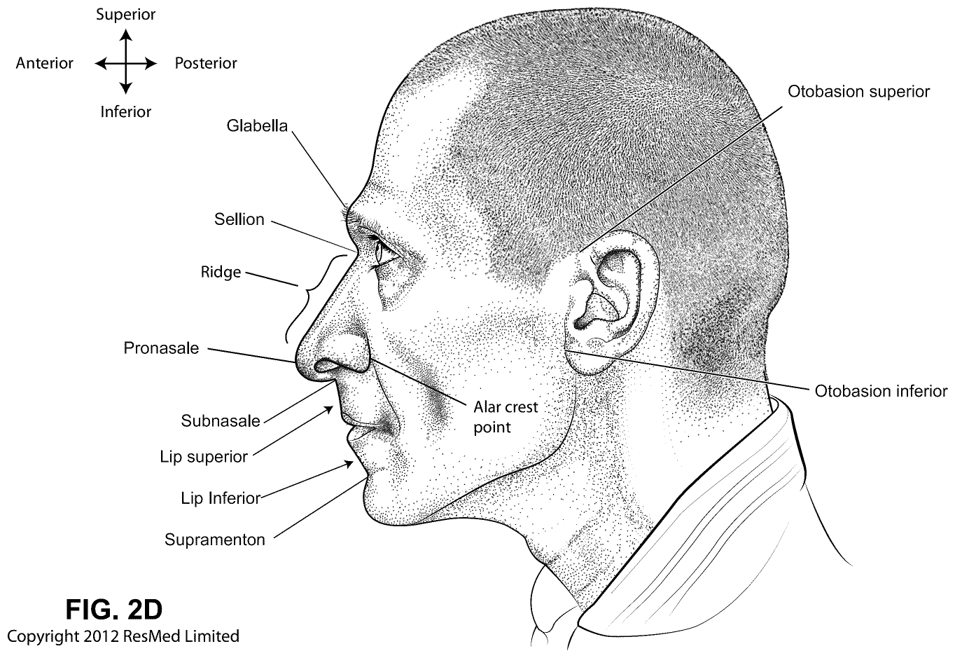

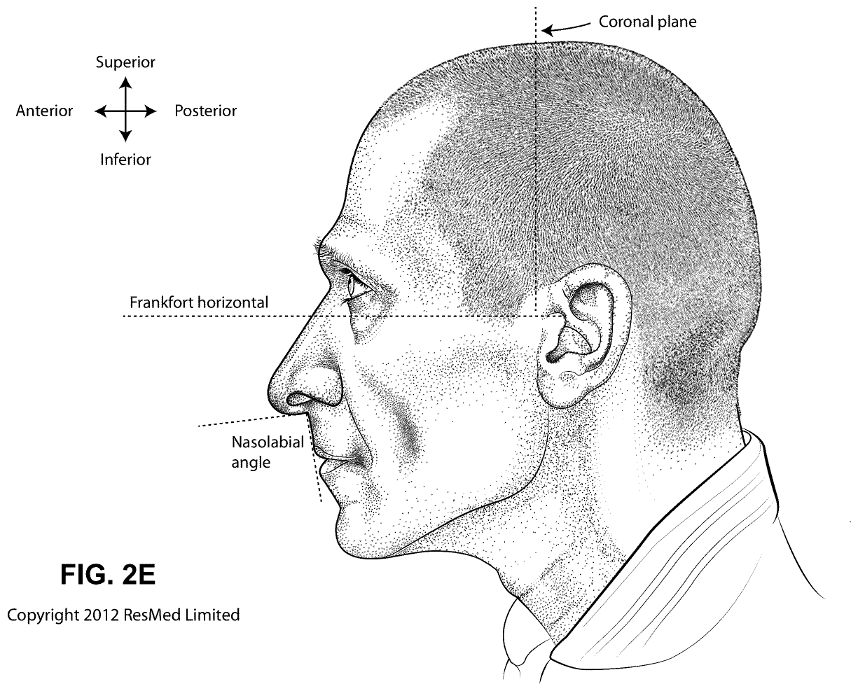

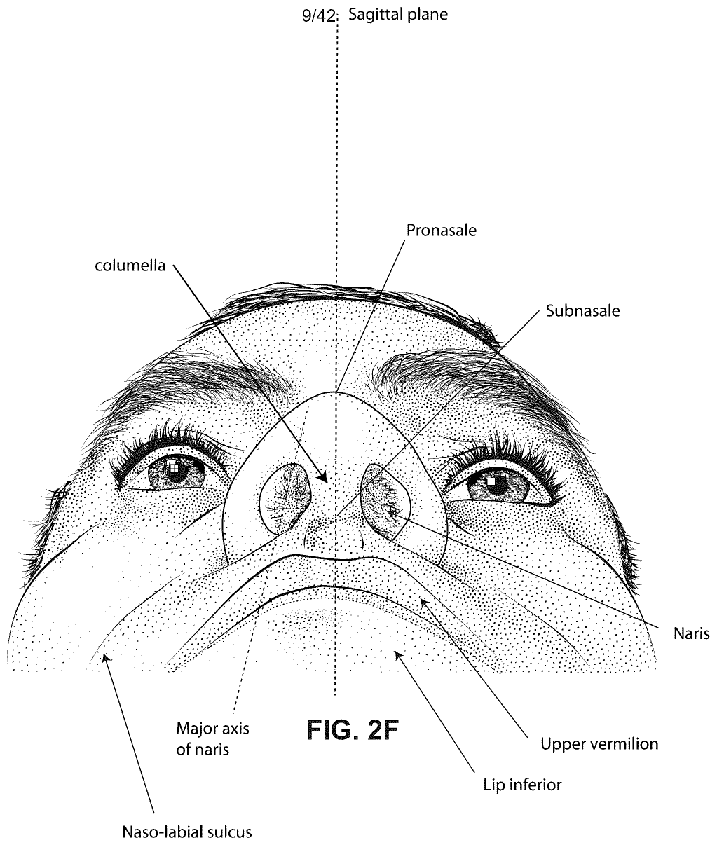

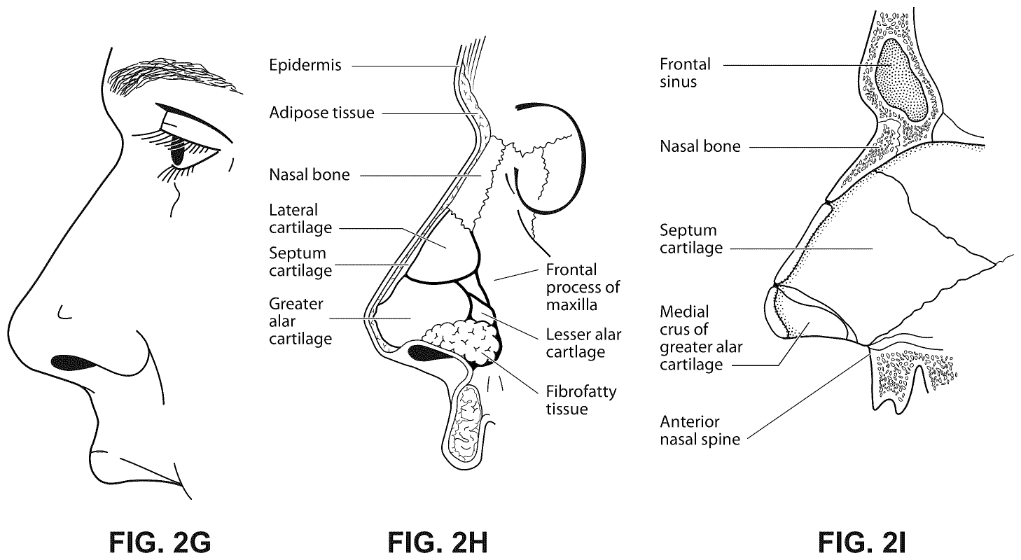

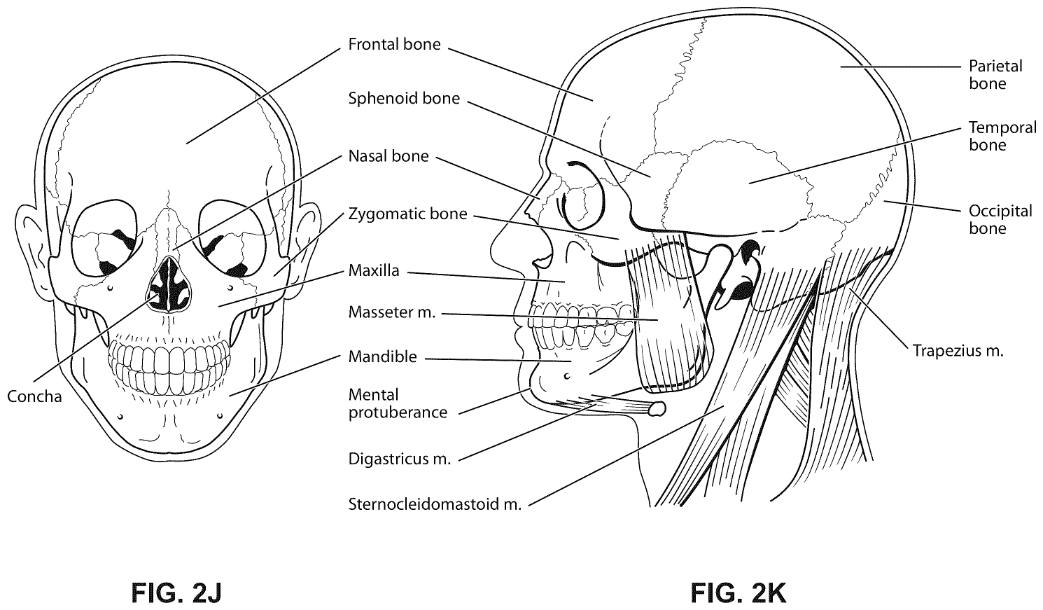

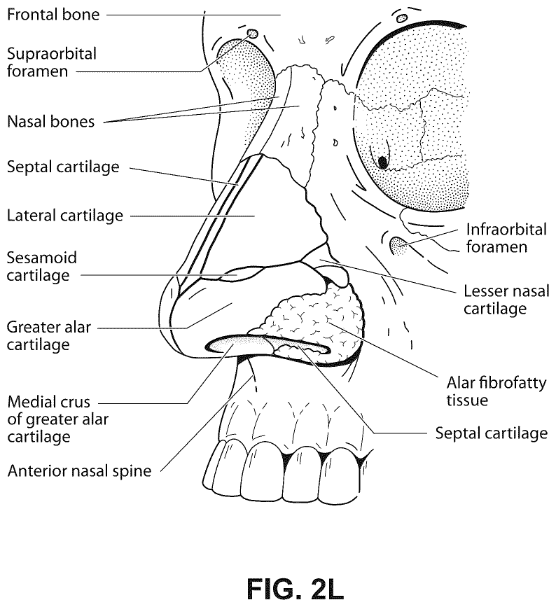

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoid and trapezius.

FIG. 2L shows an anterolateral view of a nose.

### 4.3 Patient Interface

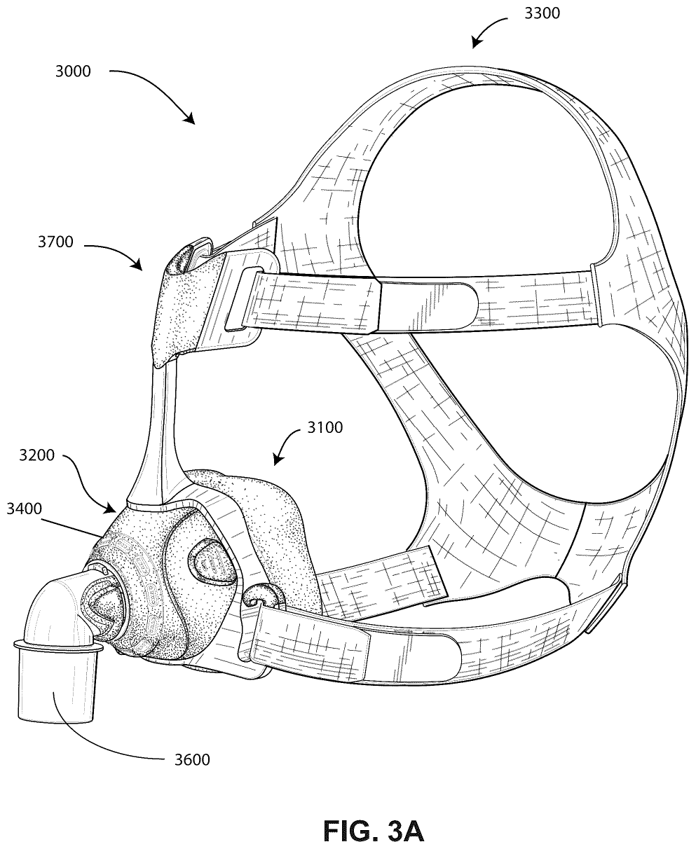

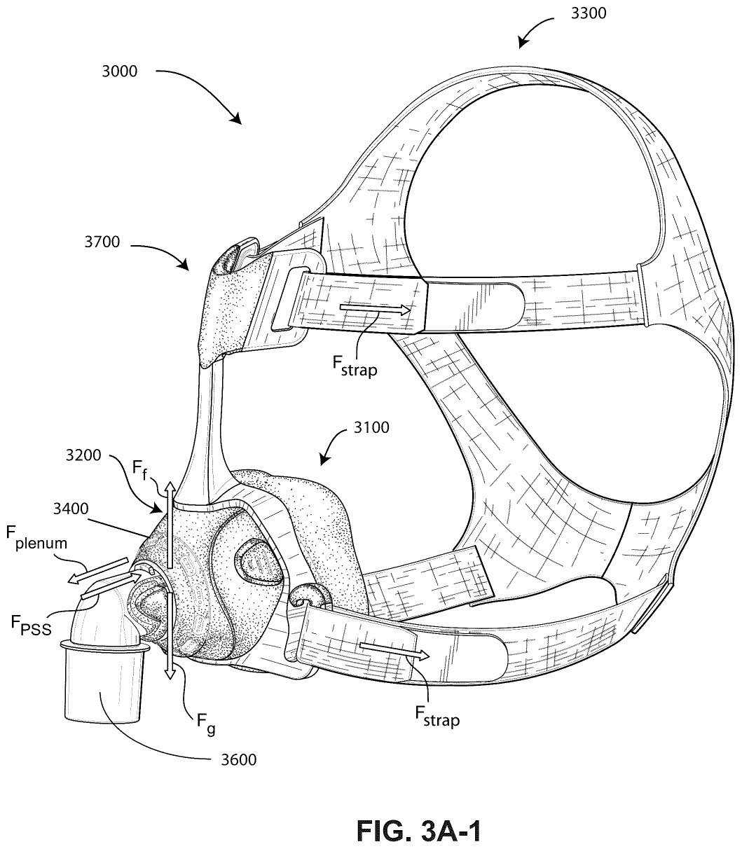

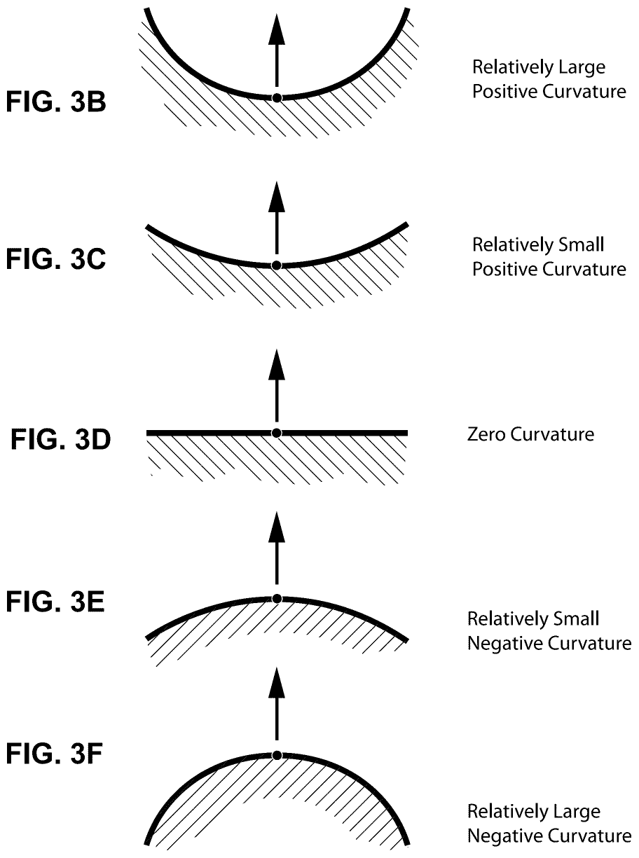

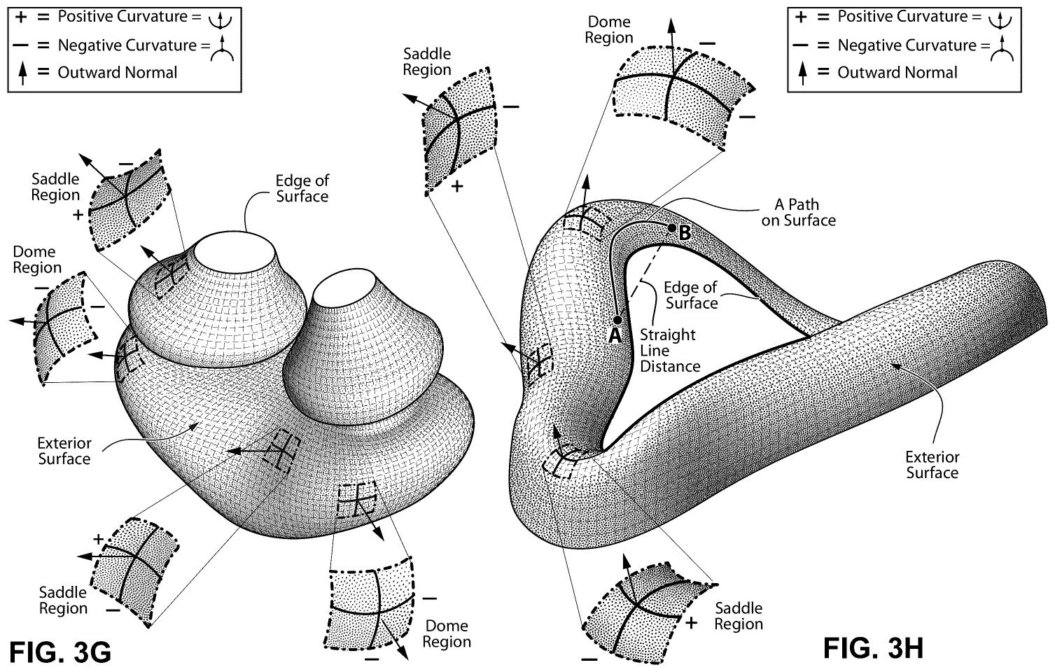

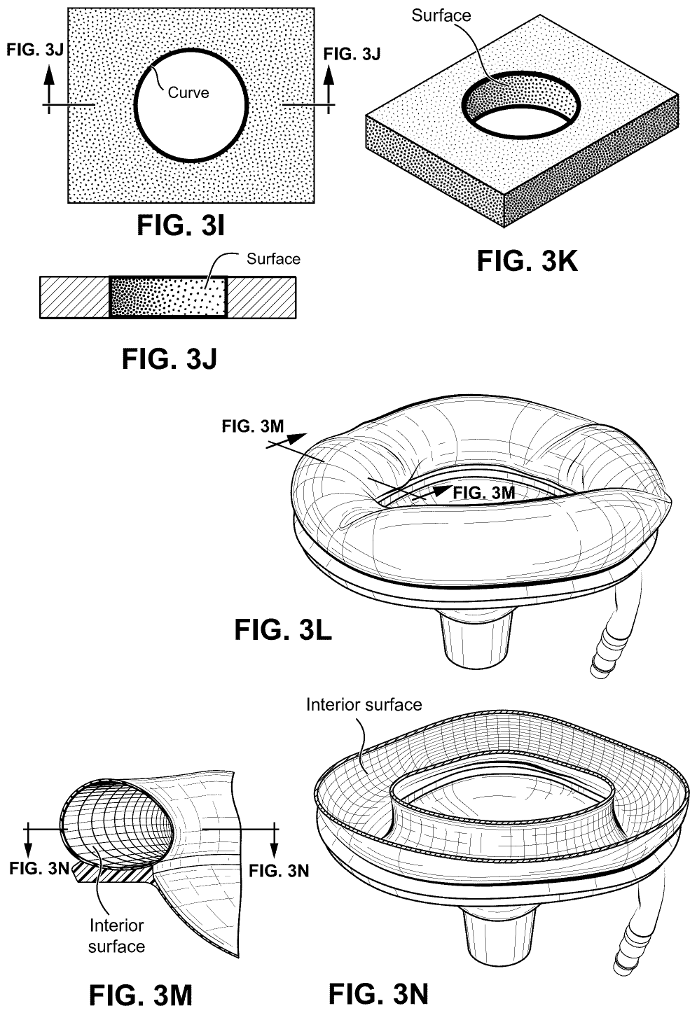

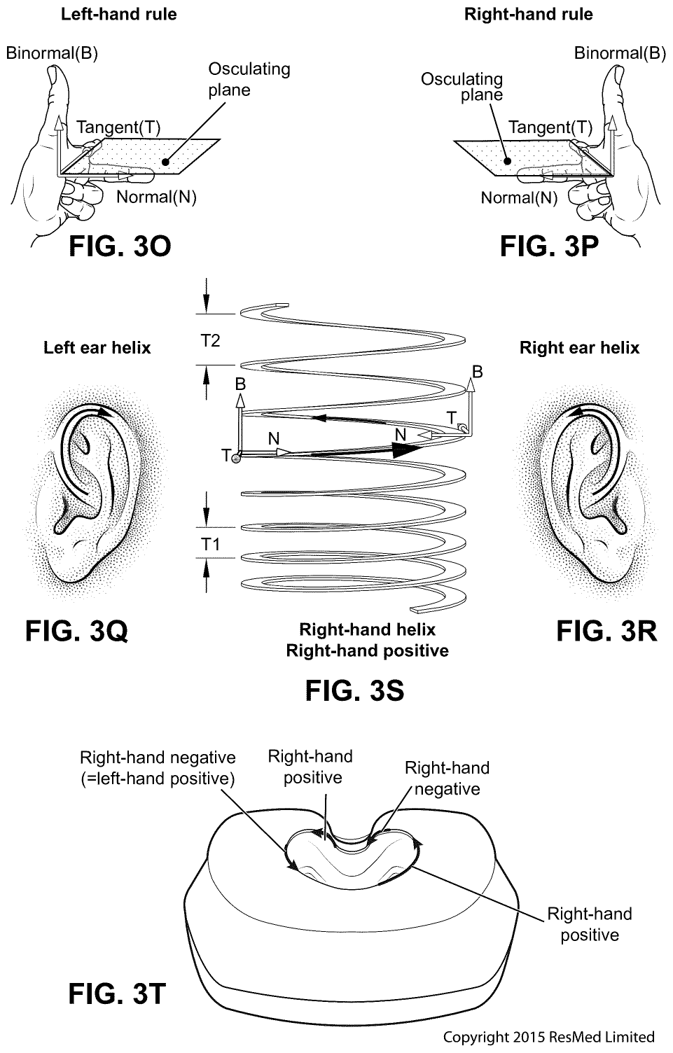

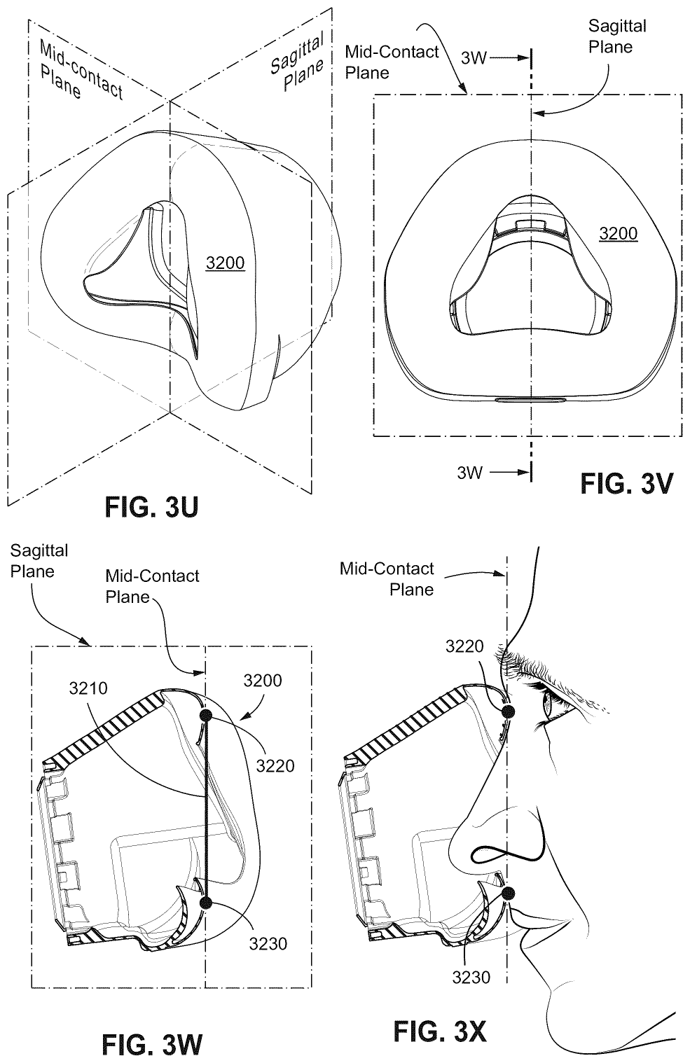

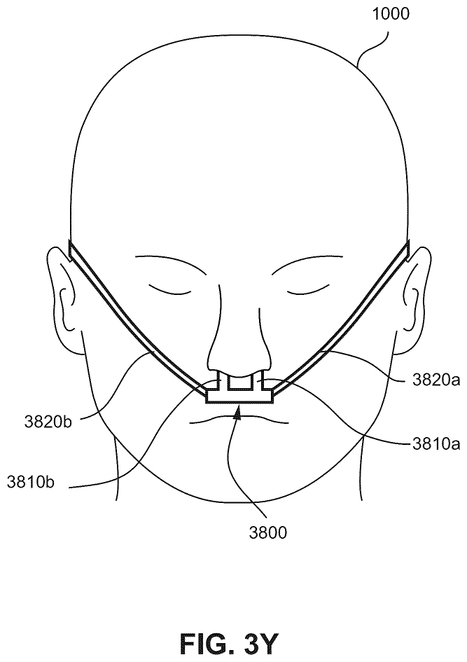

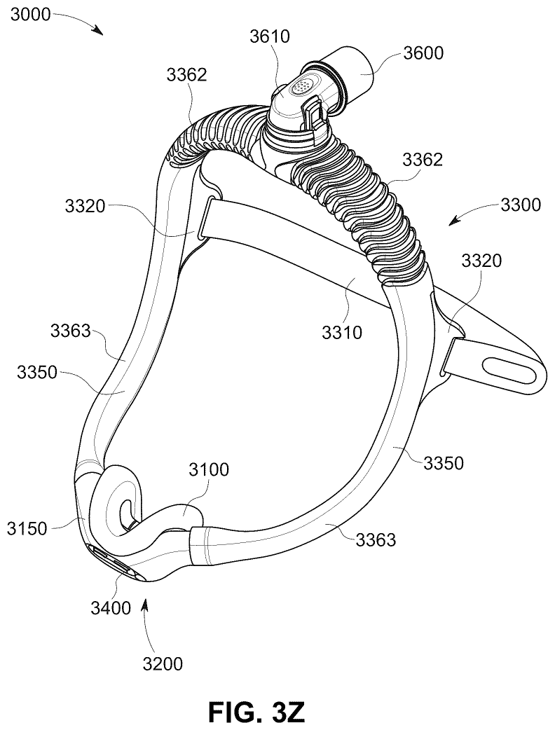

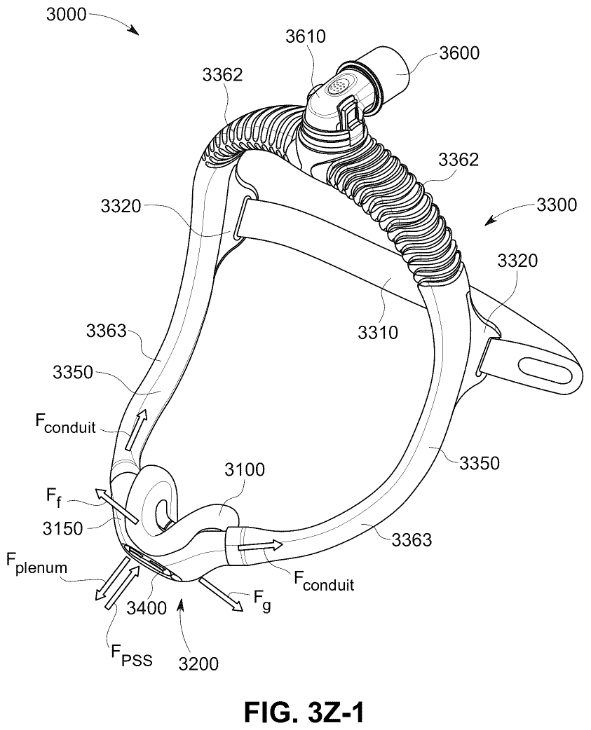

FIG. 3A shows a patient interface in the form of a nasal mask in accordance with one form of the present technology.

FIG. 3A-1 shows forces acting on the patient interface of FIG. 3A, while in use.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

FIG. 3K shows a perspective view of the structure of FIG. 3I, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3210 which lies on the sagittal plane and just touches the cushion of the plenum chamber at two points on the sagittal plane: a superior point 3220 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3220 sits approximately on the sellion, while the inferior point 3230 sits on the lip superior.

FIG. 3Y shows a patient interface in the form of a nasal cannula in accordance with one form of the present technology.

FIG. 3Z shows a patient interface having conduit headgear, in accordance with one form of the present technology.

FIG. 3Z-1 shows forces acting on the patient interface of FIG. 3Z, while in use.

### 4.4 RPT Device

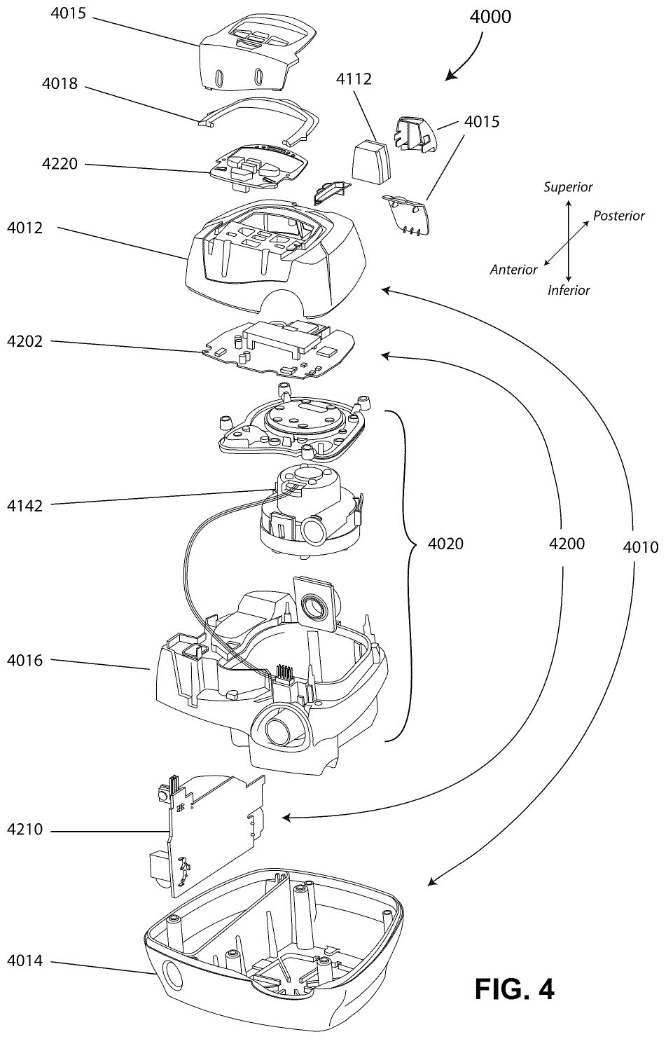

FIG. 4 shows an RPT device in accordance with one form of the present technology.

### 4.5 Humidifier

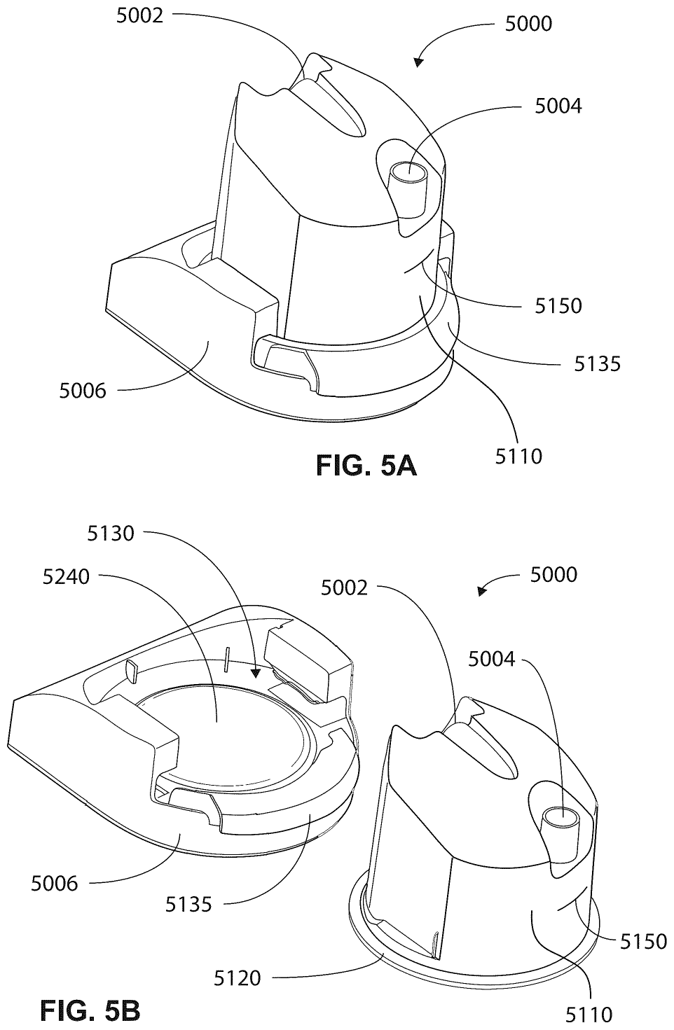

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 4.6 Breathing Waveforms

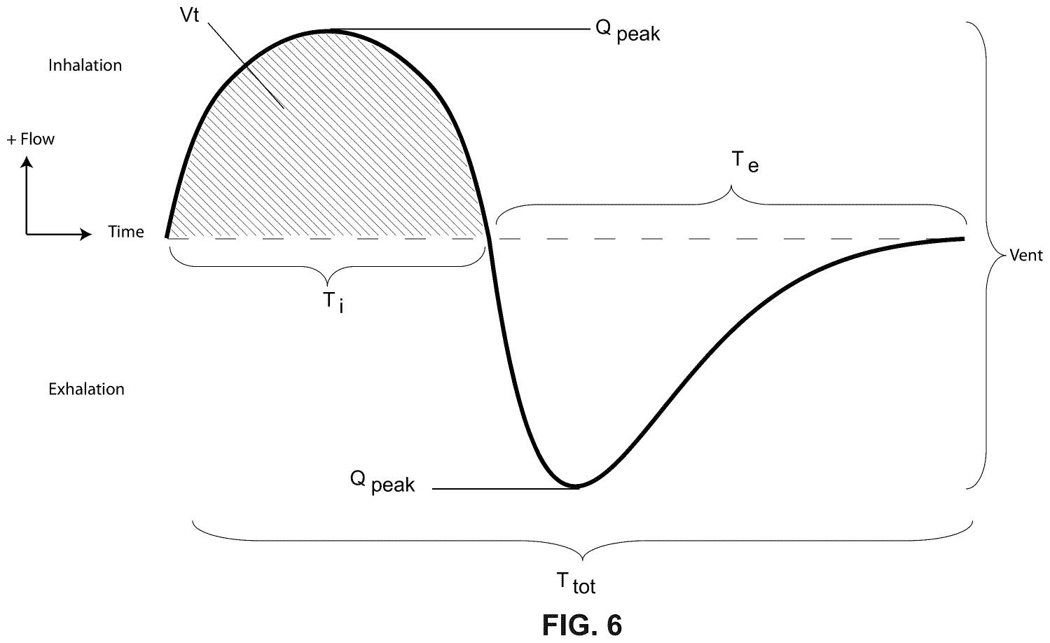

FIG. 6 shows a model typical breath waveform of a person while sleeping.

### 4.7 Modularity

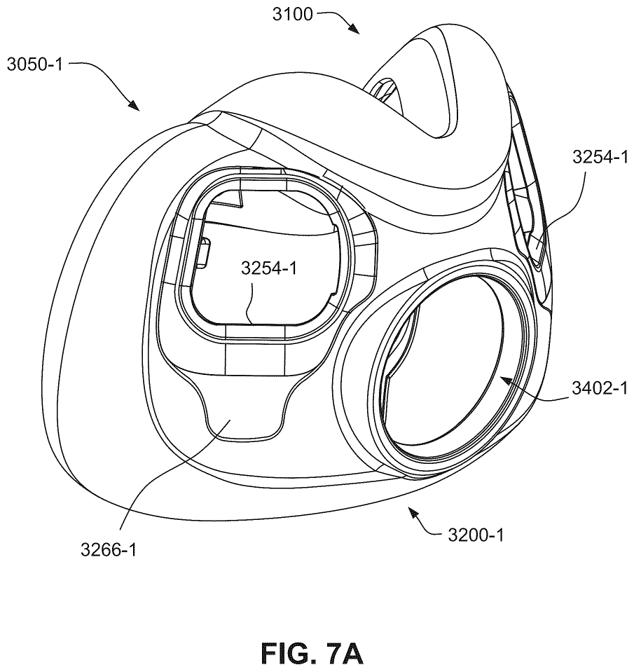

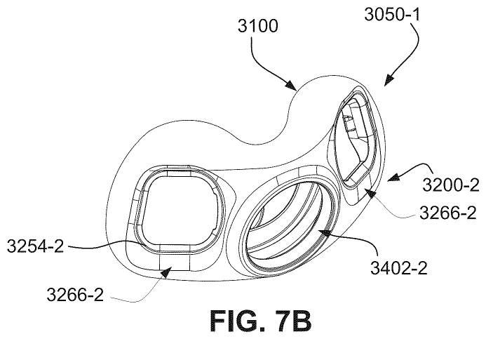

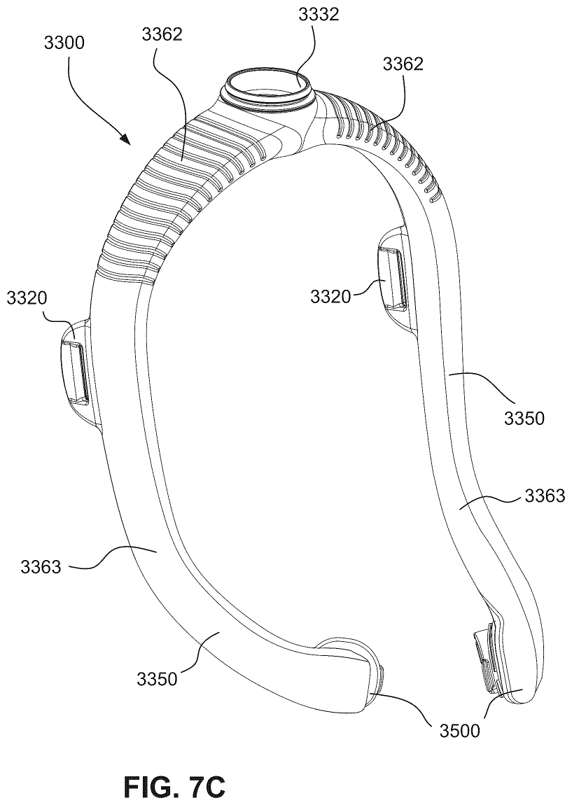

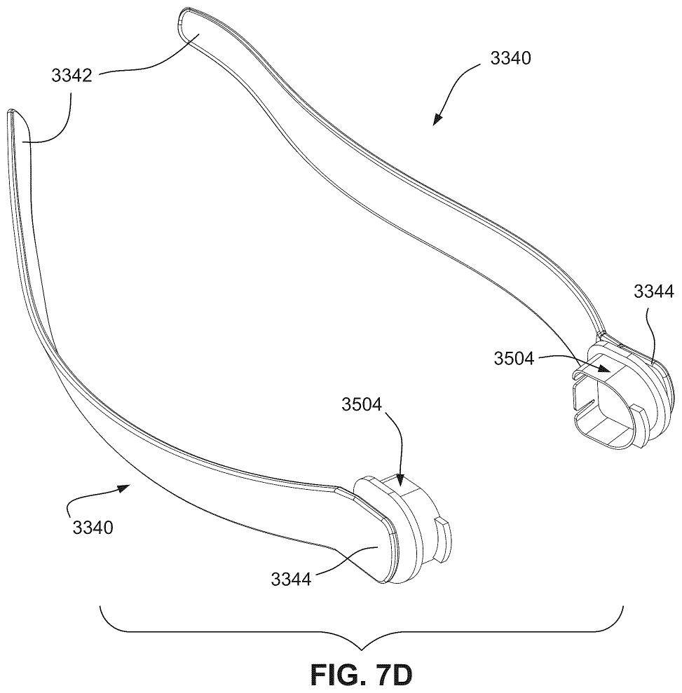

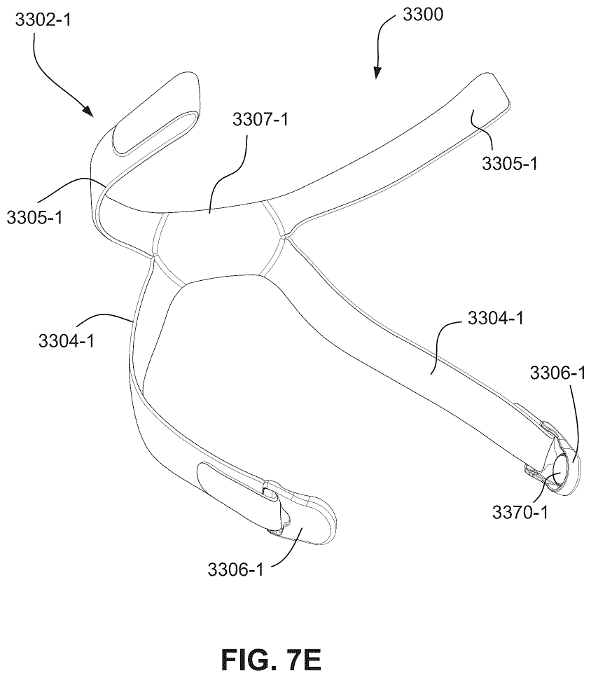

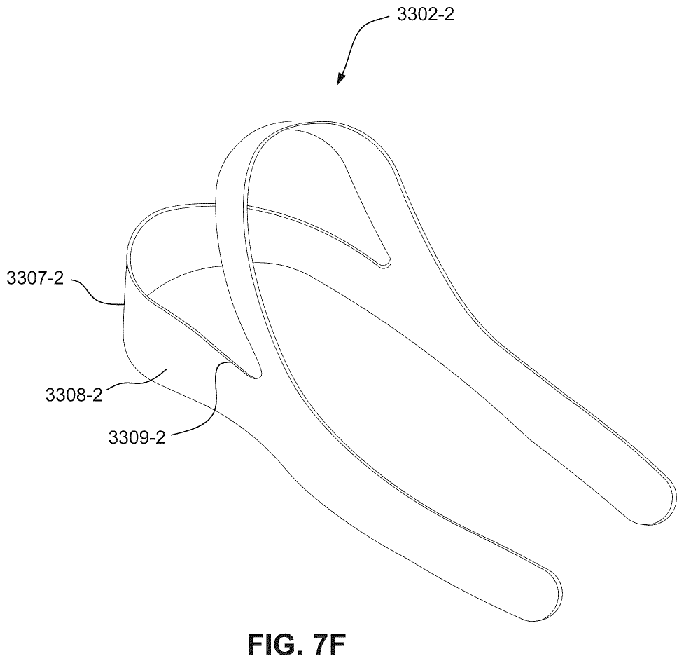

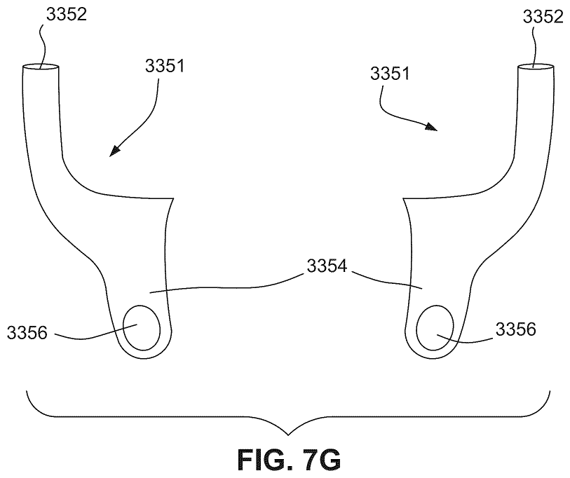

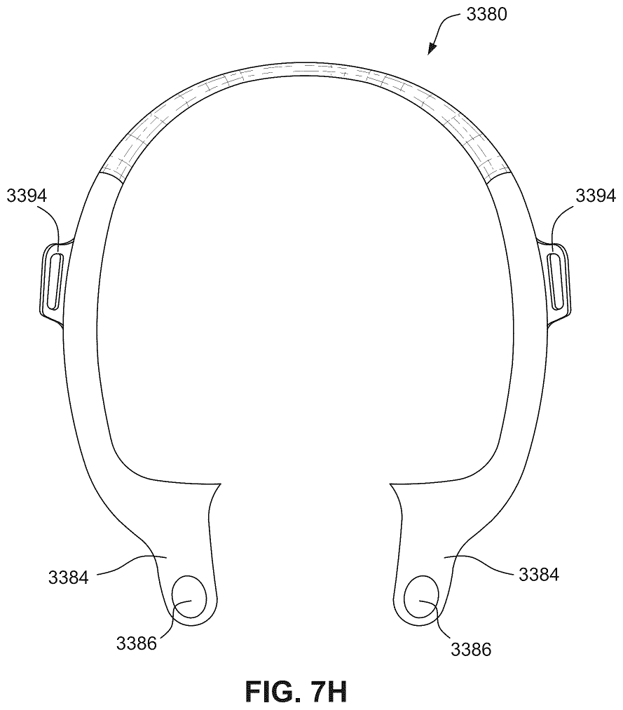

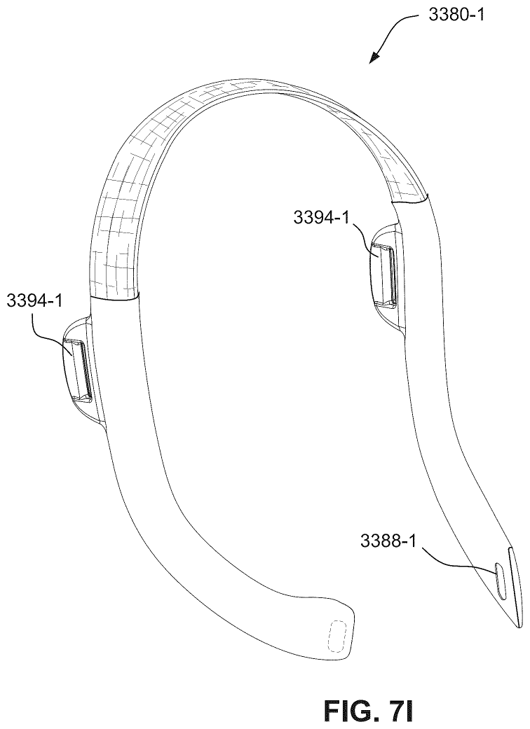

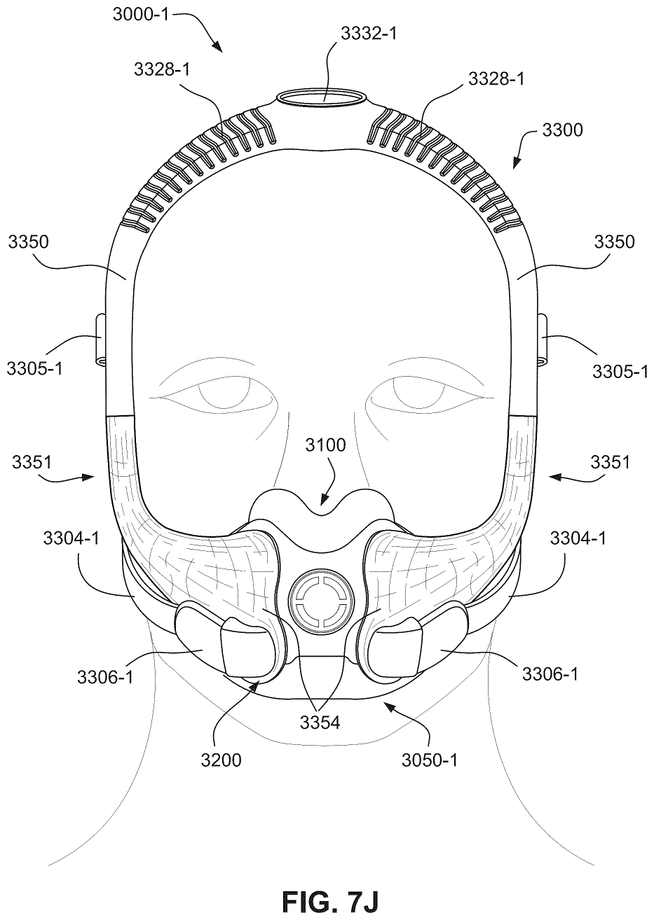

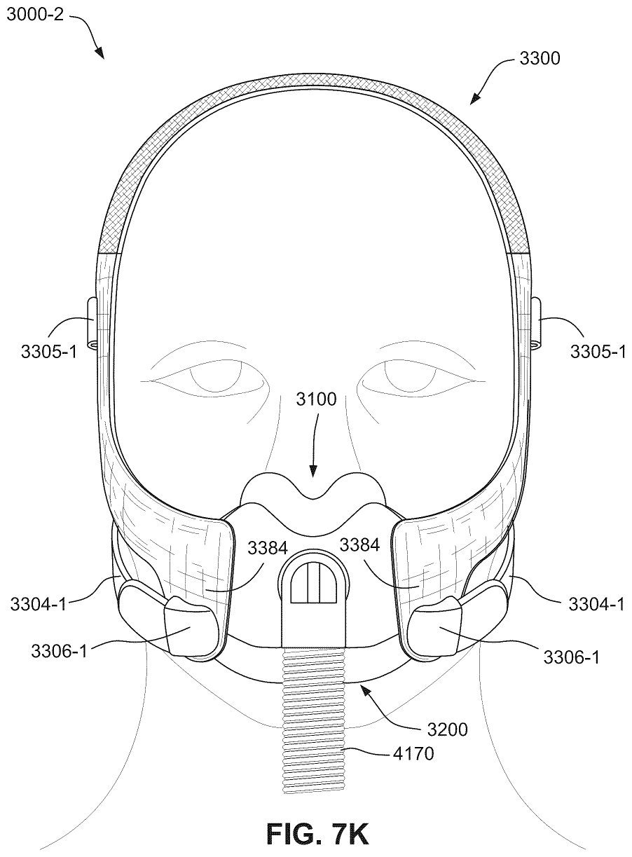

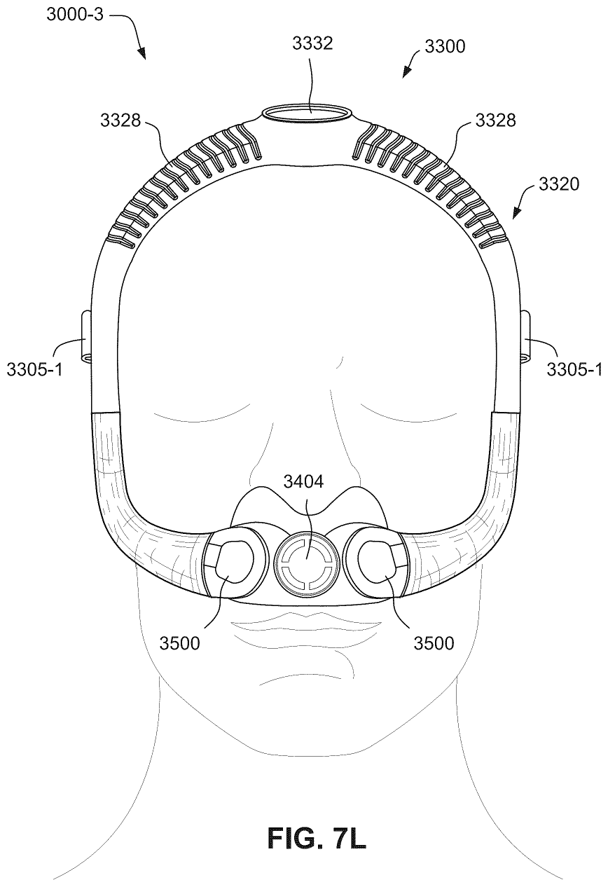

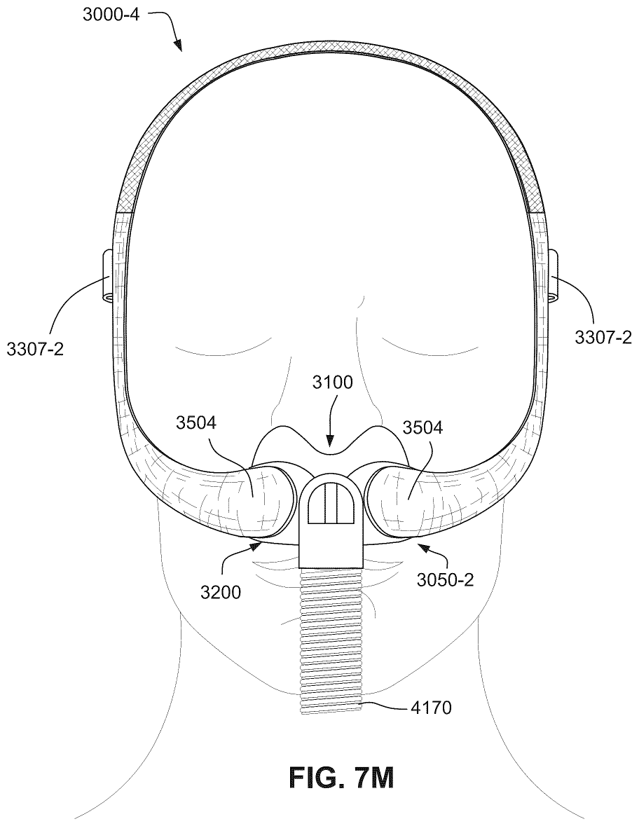

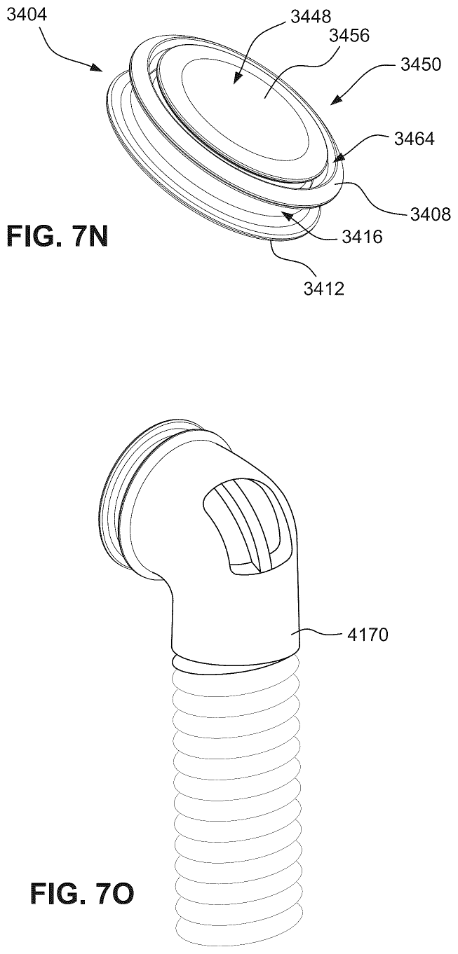

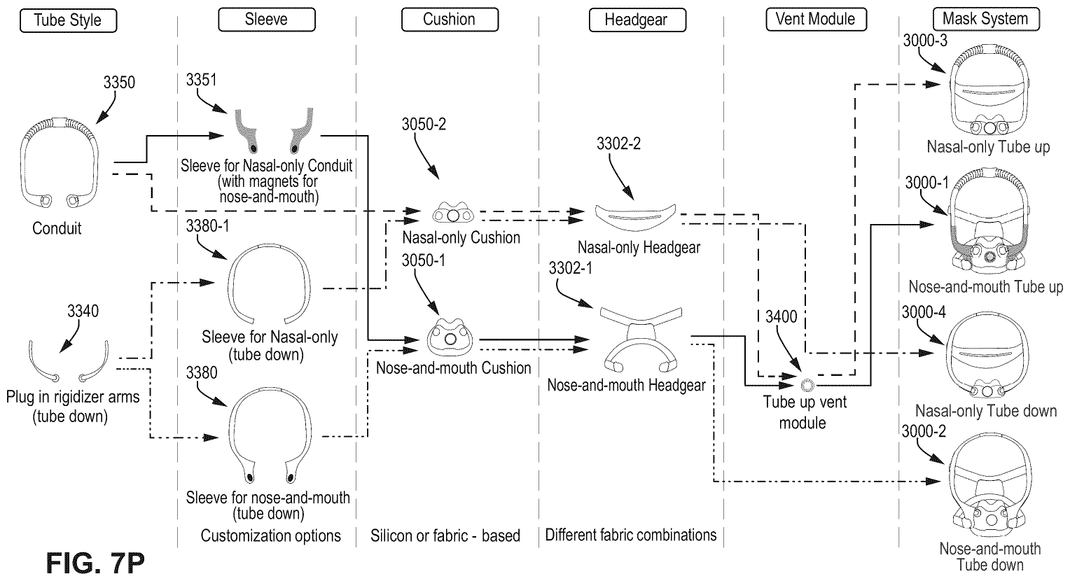

FIG. 7A shows a perspective view of a cushion of a patient interface configured to be worn by a patient and convey pressurized air to the patient's nose and the patient's mouth.

FIG. 7B shows a perspective view of a cushion of a patient interface configured to be worn by a patient and convey pressurized air to the patient's nose.

FIG. 7C shows a perspective view of tubes usable with either the cushion of FIG. 7A or the cushion of FIG. 7B.

FIG. 7D shows a perspective view of rigidiser arms usable with either the cushion of FIG. 7A of the cushion of FIG. 7B.

FIG. 7E shows a perspective view of headgear straps usable with the cushion of FIG. 7A.

FIG. 7F shows a perspective view of headgear straps usable with the cushion of FIG. 7B.

FIG. 7G shows a front view of a pair of sleeves that is removably fitted to either the tubes of FIG. 7C or the rigidiser arms of FIG. 7D.

FIG. 7H shows a front view of a full sleeve that is removably fitted to the rigidiser arms of FIG. 7D.

FIG. 7I shows a front perspective view of yet another alternate form of a full sleeve that is removably fitted to the rigidiser arms of FIG. 7D.

FIG. 7J is a front view of a patient wearing the cushion of FIG. 7A connected to the tubes of FIG. 7C, the headgear straps of FIG. 7E, and the sleeves of FIG. 7G.

FIG. 7K is a front view of a patient wearing the cushion of FIG. 7A connected to the rigidiser arms of FIG. 7D, the headgear straps of FIG. 7E, and the sleeve of FIG. 7H.

FIG. 7L is a front view of a patient wearing the cushion of FIG. 7B connected to the conduit headgear of FIG. 7C, and the headgear straps of FIG. 7F.

FIG. 7M is a front view of a patient wearing the cushion of FIG. 7B connected to the rigidisier arms of FIG. 7D, the headgear straps of FIG. 7F, and the sleeve of FIG. 7I.

FIG. 7N is an isolated perspective view of the vent of FIG. 7L.

FIG. 7O is an isolated perspective view of a portion of the air circuit of FIG. 7M.

FIG. 7P is a schematic view illustrating the possible combinations of the patient interfaces.

### 4.8 Patient Interfaces Including Positioning and Stabilising Structures of the Present Technology

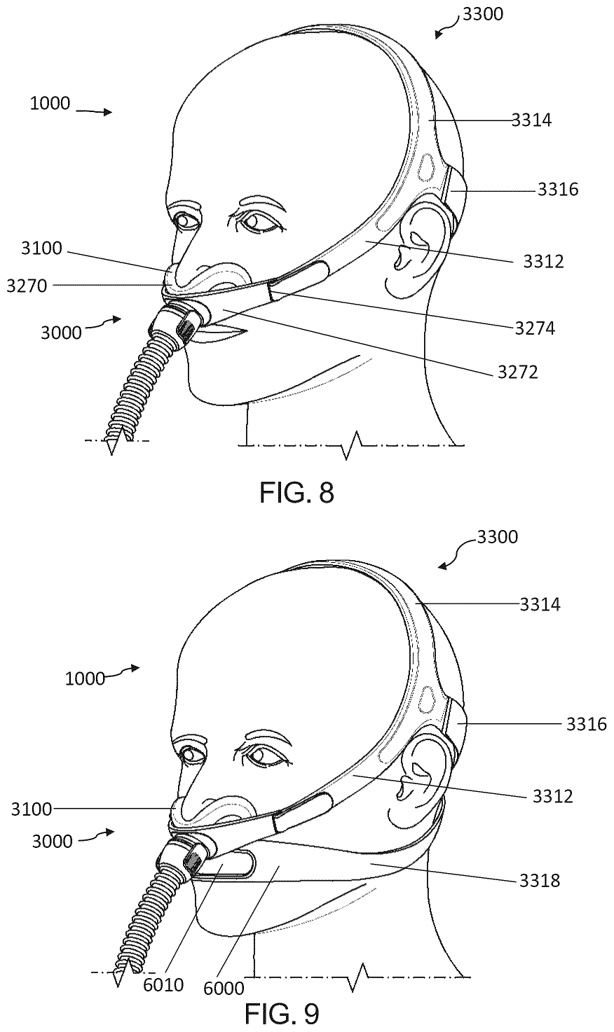

FIG. 8 is a perspective view of a patient interface with a positioning and stabilising structure according to one form of the technology.

FIG. 9 is a perspective view of a patient interface with a positioning and stabilising structure according to another form of the technology.

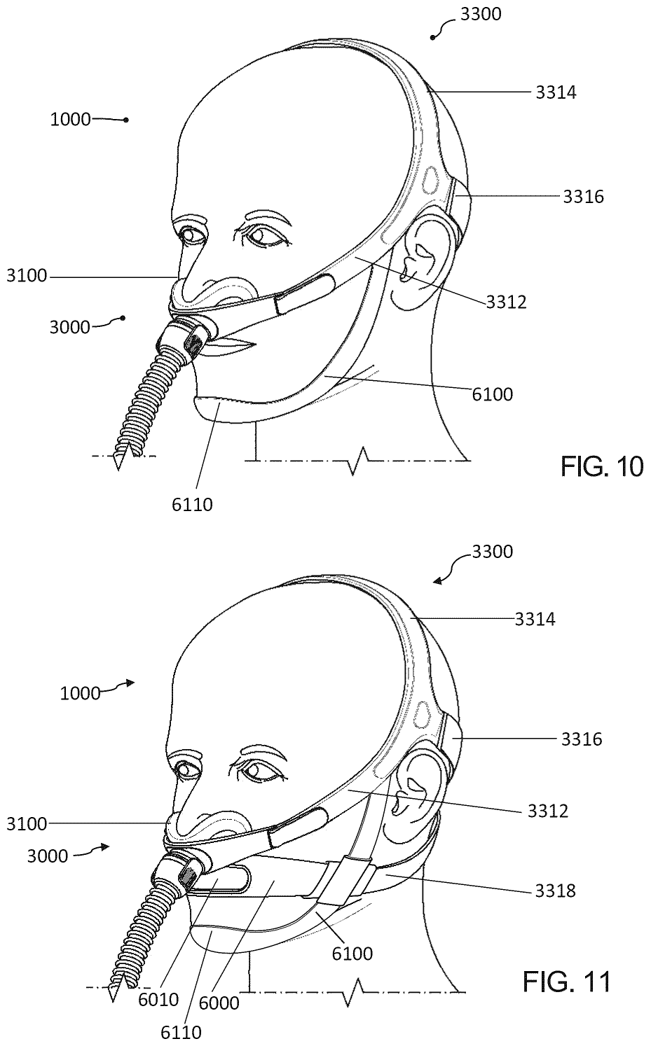

FIG. 10 is a perspective view of a patient interface with a positioning and stabilising structure according to one form of the technology.

FIG. 11 is a perspective view of a patient interface with a positioning and stabilising structure according to one form of the technology.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 5.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 3Z, a non-invasive patient interface 3000 in accordance with another aspect of the present technology comprises the following functional aspects: a seal-forming structure 3100, a plenum chamber 3200, a positioning and stabilising structure 3300, a vent 3400 and one form of connection port 3600 for connection to an air circuit (such as the air circuit 4170 shown in FIGS. 1A-IC). The plenum chamber 3200 may be formed of one or more modular components (e.g., a cushion module 3150 together with the seal-forming structure 3100) in the sense that it or they can be replaced with different components, for example components of a different size.

An unsealed patient interface 3800, in the form of a nasal cannula, includes nasal prongs 3810a, 3810b which can deliver air to respective nares of the patient 1000 via respective orifices in their tips. Such nasal prongs do not generally form a seal with the inner or outer skin surface of the nares. This type of interface results in one or more gaps that are present in use by design (intentional) but they are typically not fixed in size such that they may vary unpredictably by movement during use. This can present a complex pneumatic variable for a respiratory therapy system when pneumatic control and/or assessment is implemented, unlike other types of mask-based respiratory therapy systems. The air to the nasal prongs may be delivered by one or more air supply lumens 3820a, 3820b that are coupled with the nasal cannula-type unsealed patient interface 3800. The lumens 3820a, 3820b lead from the nasal cannula-type unsealed patient interface 3800 to a respiratory therapy device via an air circuit. The unsealed patient interface 3800 is particularly suitable for delivery of flow therapies, in which the RPT device generates the flow of air at controlled flow rates rather than controlled pressures. The “vent” or gap at the unsealed patient interface 3800, through which excess airflow escapes to ambient, is the passage between the end of the prongs 3810a and 3810b of the nasal cannula-type unsealed patient interface 3800 via the patient's nares to atmosphere.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient, for example at least 2, 4, 6, 10, or 20 cmH2O with respect to ambient.

### 5.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 5.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 5.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 5.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 5.3.1.5 Forehead Region

In one form, the seal-forming structure that forms a seal in use on a forehead region of the patient's face. In such a form, the plenum chamber may cover the eyes in use.

### 5.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

### 5.3.1.7 Nose-Only Masks

In one form, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form, for example as shown in FIG. 3Z, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

In some forms, a nose-only mask may comprise nasal pillows, described above.

### 5.3.1.8 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 3100 configured to seal on the patient's face around the nose, below the mouth and over the bridge of the nose. A nose-and-mouth mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “nose-and-mouth cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

### 5.3.2 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

In some forms, the plenum chamber 3200 is constructed from a rigid material such as polycarbonate. The rigid material may provide support to the seal-forming structure.

In some forms, the plenum chamber 3200 is constructed from a flexible material (e.g., constructed from a soft, flexible, resilient material like silicone, textile, foam, etc.). For example, in examples then may be formed from a material which has a Young's modulus of 0.4 GPa or lower, for example foam. In some forms of the technology the plenum chamber 3200 may be made from a material having Young's modulus of 0.1 GPa or lower, for example rubber. In other forms of the technology the plenum chamber 3200 may be made from a material having a Young's modulus of 0.7 MPa or less, for example between 0.7 MPa and 0.3 MPa. An example of such a material is silicone.

### 5.3.2.1 Multiple Openings

As shown in FIGS. 7A and 7B, different plenum chambers 3200-1, 3200-2 may be formed as part of a multi-opening cushion 3050-1, 3050-2. In the illustrated examples, the cushions 3050-1, 3050-2 each include three openings, although an alternate cushion may be formed with greater or fewer openings.

In some forms, the different openings may serve different functions. For example, some openings may be exclusively inlet openings, while other openings may be exclusively outlet openings.

In other forms, at least one opening may serve two different functions. For example, one opening may operate as both an inlet and an outlet during the same breathing cycle.

The plurality of openings may allow for a variety of configurations of air delivery to the plenum chamber 3200-1, 3200-2. For example, depending on patient need and/or patient comfort, the patient may use a given cushion 3050-1, 3050-2 in a “tube-up” configuration (e.g., using conduit headgear—described below) or a “tube-down” configuration (e.g., using a single conduit in front of the patient's face).

### 5.3.2.1.1 Nose and Mouth Mask

As shown in FIG. 7A, the plenum chamber 3200-1 includes a pair of plenum chamber inlet ports 3254-1, which may be used to convey gas into and/or out of the plenum chamber 3200-1. The plenum chamber inlet ports 3254-1 may be disposed on opposite sides (e.g., left and right sides) of the plenum chamber 3200-1.

In some forms, the plenum chamber 3200-1 may also include at least one vent opening 3402-1 (see e.g., FIG. 7A). The vent opening 3402-1 may be disposed in a center of the plenum chamber 3200-1. For example, the vent opening 3402-1 may be disposed between the plenum chamber inlet ports 3254-1.

In some forms, the plenum chamber 3200-1 may include a pair of grooves 3266-1. Each groove 3266-1 may be disposed proximate to one of the plenum chamber inlet ports 3254-1. Each groove 3266-1 may form a partially recessed surface.

### 5.3.2.1.2 Nose-Only Mask

The plenum chamber 3200-2 of a nasal only cushion 3050-2 may be similar to the plenum chamber 3200-1 of the mouth and nose cushion 3050-1. Only some similarities and differences between the plenum chambers 3200-1, 3200-2 may be described below.

As shown in FIG. 7B, the plenum chamber 3200-2 includes a pair of plenum chamber inlet ports 3254-2, which may be used to convey gas into and/or out of the plenum chamber 3200-2. The plenum chamber inlet ports 3254-2 may be disposed on opposite sides (e.g., left and right sides) of the plenum chamber 3200-2.

In some forms, the plenum chamber 3200-2 may also include at least one vent opening 3402-2 (see e.g., FIG. 7B). The vent opening 3402-2 may be disposed in a center of the plenum chamber 3200-2. For example, the vent opening 3402-2 may be disposed between the plenum chamber inlet ports 3254-2.

In some forms, the plenum chamber 3200-2 may include a pair of grooves 3266-2. Each groove 3266-2 may be disposed proximate to one of the plenum chamber inlet ports 3254-2. Each groove 3266-2 may form a partially recessed surface.

### 5.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position. Examples of a positioning and stabilising structure may be shown in FIGS. 3A and 3A-1.

In one form the positioning and stabilising structure 3300 provides a retention force at least sufficient to overcome the effect of the positive pressure in the plenum chamber 3200 to lift off the face (i.e., Fₚₗₑₙᵤₘ).

In one form the positioning and stabilising structure 3300 provides a retention force to overcome the effect of the gravitational force on the patient interface 3000.

With continued reference to FIG. 3A-1, the positioning and stabilising structure 3300 provides a force F_{PSS} that assists in maintaining the plenum chamber 3200 in the sealing position on the patient's face. The positioning and stabilising force F_{PSS} may be the resultant force from the various forces of the different elements of the positioning and stabilising structure 3300. For example, headgear straps may individually provide a strap force Fₛₜᵣₐₚ in order to hold the seal-forming structure 3100 against the patient's face. The force Fₛₜᵣₐₚ may also be directed at least partially in the superior direction in order to overcome the gravitational force F_{g}. The gravitational force F_{g} may be specifically shown for the seal-forming structure 3100 and the plenum chamber 3200, but gravity would act on the entirely of the patient interface 3000 (i.e., in the same direction as the illustrated gravitational force F_{g}).

The gravitational force F_{g} may be opposed by a frictional force F_{f}, which may act in a direction directly opposite of the gravitational force F_{g}. As gravity pulls the seal-forming structure 3100 and the plenum chamber 3200 in the inferior direction (as viewed in FIG. 3A-1), the frictional force F_{f} would act in the superior direction (e.g., against a patient's face). For example, the patient may experience the frictional force F_{f} against his lip superior (and/or other surfaces of the patient's face in contact with the seal-forming structure 3100) in order to oppose the motion in the inferior direction (which may help to stabilising the cushion in place). Although the frictional force F_{f} is shown specifically opposing the gravitational force F_{g} of the seal-forming structure 3100 and the plenum chamber 3200, components of an overall frictional force (not shown) would also oppose the gravitational force F_{g} associated with the positioning and stabilising structure 3300 and any other portions of the patient interface 3000. A force of friction can act along any place where the patient interface 3000 contacts the patient's skin (or hair). The frictional force F_{f} extends in the opposite direction of the gravitational force F_{g} and along the patient's skin (or hair). In some forms the gravitational force F_{g} may also be countered by vertical components of the reaction force from the patient's face acting on the seal-forming structure 3100, for example at the nose ridge and chin regions of the patient's face, for example.

In some forms, the sum of the various forces may equal zero so that the patient interface 3000 is at equilibrium (e.g., not moving along the patient's face while in use). Specifically, the gravitational force F_{g} and the blowout force Fₚₗₑₙᵤₘ tend to move the seal-forming structure 3100 away from the desired sealing position. The positioning and stabilising force F_{PSS} is applied in order to counteract the gravitational force F_{g} and the blowout force Fₚₗₑₙᵤₘ (as well as any frictional forces F_{f}) and keep the seal-forming structure 3100 properly situated. Although the positioning and stabilising force F_{PSS} may exceed the sum of the gravitational force F_{g} and the blowout force Fₚₗₑₙᵤₘ (with any additional positioning and stabilising force F_{PSS} being balanced by reaction force from the patient's head acting on the portions of patient interface 3000) and still maintain the seal-forming structure 3100 in an appropriate sealing position, patient comfort may be sacrificed. Maximum patient comfort may be achieved when the net force on the patient interface 3000 is zero and the positioning and stabilising force F_{PSS} is exactly strong enough to achieve this. In some examples the positioning and stabilising structure 3300 may be adjustable such that when fitted the positioning and stabilising force F_{PSS} is greater than required to exactly balance the gravitational force F_{g} and the blowout force Fₚₗₑₙᵤₘ to hold the patient interface 3000 against the patient's head tightly enough that disruptive forces which may be experienced in use (such as tube drag or lateral shunting of the plenum chamber 3200 during side sleeping) do not disrupt the seal. As described below, various positions of the patient's head while using the patient interface 3000 may determine the positioning and stabilising force F_{PSS} necessary to achieve equilibrium.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilising structure 3300, each being configured to provide a retaining force to correspond to a different size and/or shape range. For example the system may comprise one form of positioning and stabilising structure 3300 suitable for a large sized head, but not a small sized head, and another. suitable for a small sized head, but not a large sized head.

### 5.3.3.1 Conduit Headgear

### 5.3.3.1.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3Z, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

In the form of the present technology illustrated in FIG. 3Z, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes.

In one example in which the patient interface has one tube 3350, the single tube 3350 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head. For example, the tube 3350 and the strap may each be under tension in use in order to assist in maintaining the seal-forming structure 3100 in a sealing position.

In one form, the tube 3350 may be at least partially extensible so that the tube 3350 and the strap may adjust substantially equal lengths when worn by a patient. This may allow for substantially symmetrical adjustments between the tube 3350 and the strap so that the seal-forming structure remains substantially in the middle.

In the form of the technology shown in FIG. 3Z, the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used, they may be indirectly connected together, for example each may be connected to a T-shaped connector. The T-shaped connector may have two arms/branches each fluidly connectable to a respective one of the tubes 3350. Additionally, the T-shaped connector may have a third arm or opening providing the connection port 3600 for fluid connection to the air circuit 4170 in use. The opening may be an inlet 3332 (see e.g., 7C) for receiving the flow of pressurized air.

In some forms, the third arm of the T-shaped connector may be substantially perpendicular to each of the first two arms.

In some forms, the third arm of the T-shaped connector may be obliquely formed with respect to each of the first two arms.

In some forms, a Y-shaped connector may be used instead of the T-shaped connector. The first two arms may be oblique with respect to one another, and the third arm may be oblique with respect to the first two arms. The angled formation of the first two arms may be similar to the shape of the patient's head in order to conform to the shape.

In some forms, at least one of the arms of the T-shaped connector (or Y-shaped connector) may be flexible. This may allow the connector to bend based on the shape of the patient's head and/or a force in the positioning and stabilising structure 3300.

In some forms, at least one of the arms of the T-shaped connector (or Y-shaped connector) may be at least partially rigidised. This may assist in maintaining the shape of the connector so that bending of the connector does not close the airflow path.

The tubes 3350 may be formed from a flexible material, such as an elastomer, e.g. silicone or TPE, and/or from one or more textile and/or foam materials. The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

In some examples, the one or more tubes 3350 are crush resistant to resist being blocked if crushed during use, for example if squashed between a patient's head and pillow, especially if there is only one tube 3350. The tubes 3350 may be formed with a sufficient structural stiffness to resist crushing or may be as described in U.S. Pat. No. 6,044,844, the contents of which are incorporated herein by reference.

Each tube 3350 may be configured to receive a flow of air from the connection port 3600 on top of the patient's head and to deliver the flow of air to the seal-forming structure 3100 at the entrance of the patient's airways. In the example shown in FIG. 3Z, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3350 are configured to allow movement of an upper portion of the patient interface 3000 (e.g. a connection port 3600) with respect to a lower portion of the patient interface 3000 (e.g. a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force in a posterior and superior direction (e.g. a posterosuperior direction). A sealing force with a posterosuperior direction may cause the seal-forming structure 3100 to form a good seal to both the inferior periphery of the patient's nose and anterior-facing surfaces of the patient's face, for example on either side of the patient's nose and the patient's lip superior.

Conduits forming part of the positioning and stabilising structure 3300, like headgear straps, may provide a force that contributes to the positioning and stabilising force F_{PSS}. As illustrated in FIG. 3Z-1, the positioning and stabilising force F_{PSS} may be the resultant force from the various forces of the different elements of the positioning and stabilising structure 3300. For example, each conduit may provide a force Fconduit directed in the posterior and respective lateral direction in order to hold the seal-forming structure 3100 against the patient's face (into the upper lip and sealing under the nose) and oppose the effect of the positive pressure in the plenum chamber 3200 to lift off the face (i.e., Fplenum). The force Fconduit directed may also be directed at least partially in the superior direction in order to overcome the gravitational force Fg.

In some forms, the conduits may provide a force directed into the patient's head when the conduits are filled with pressurized air. The force may assist in gripping the patient's head. The force may be caused by the inflation of the conduits during normal use. In some forms, the force may provide a cushioning effect to the patient's head. The conduits may be designed in order to limit expansion in order to prevent over-gripping the patient's head.

The position of the patient's head may also change the gripping force of the conduits. For example, if the patient is sleeping on his side, the weight of the patient's head may compress one conduit, and the other conduit (e.g., the lateral portion not between the patient's head and a sleeping surface, like a pillow) may additionally expand in order to keep substantially the same flow rate of pressurized air.

The gravitational force F_{g} may be opposed by a frictional force F_{f}, which may act in a direction directly opposite of the gravitational force F_{g}. As gravity pulls the seal-forming structure 3100 and the plenum chamber 3200 in the inferior direction (as viewed in FIG. 3A-1), the frictional force F_{f} would act in the superior direction (e.g., against a patient's face). For example, the patient may experience the frictional force F_{f} against his lip superior (and/or other surfaces of the patient's face in contact with the seal-forming structure 3100) in order to oppose the motion in the inferior direction (which may help to stabilising the cushion in place). Although the frictional force F_{f} is shown specifically opposing the gravitational force F_{g} of the seal-forming structure 3100 and the plenum chamber 3200, components of an overall frictional force (not shown) would also oppose the gravitational force F_{g} associated with the positioning and stabilising structure 3300 and any other portions of the patient interface 3000. A force of friction can act along any place where the patient interface 3000 contacts the patient's skin (or hair). The frictional force F_{f} extends in the opposite direction of the gravitational force F_{g} and along the patient's skin (or hair).

In some forms, the sum of the various forces may equal zero so that the patient interface 3000 is at equilibrium (e.g., not moving along the patient's face while in use). Specifically, the gravitational force Fg and the blowout force Fplenum tend to move the seal-forming structure 3100 away from the desired sealing position. The positioning and stabilising force F_{PSS} is applied in order to counteract the gravitational force Fg and the blowout force Fplenum (as well as any frictional forces Ff) and keep the seal-forming structure 3100 properly situated. Although the positioning and stabilising force F_{PSS} may exceed the sum of the gravitational force Fg and the blowout force Fplenum (with any additional positioning and stabilising force FPSS being balanced by reaction force from the patient's head acting on the portions of patient interface 3000) and still maintain the seal-forming structure 3100 in an appropriate sealing position, patient comfort may be sacrificed. Maximum patient comfort may be achieved when the net force on the patient interface 3000 is zero and the positioning and stabilising force F_{PSS} is exactly strong enough to achieve this. In some examples the positioning and stabilising structure 3300 may be adjustable such that when fitted the positioning and stabilising force F_{PSS} is greater than required to exactly balance the gravitational force Fg and the blowout force Fplenum to hold the patient interface 3000 against the patient's head tightly enough that disruptive forces which may be experienced in use (such as tube drag or lateral shunting of the plenum chamber 3200 during side sleeping) do not disrupt the seal. As described below, various positions of the patient's head while using the patient interface 3000 may determine the positioning and stabilising force FPSS necessary to achieve equilibrium

### 5.3.3.1.2 Extendable and Non-Extendable Tube Portions

In some examples of the present technology, one or both of the tubes 3350 are not extendable in length. However, in some forms, the tubes 3350 may comprise one or more extendable tube sections, for example formed by an extendable concertina structure. In some forms, the patient interface 3000 may comprise a positioning and stabilising structure 3300 including at least one gas delivery tube comprising a tube wall having an extendable concertina structure. The patient interface 3000 shown in FIG. 3Z comprises tubes 3350, the superior portions of which comprise extendable tube sections each in the form of an extendable concertina structure 3362.

In some forms, the extendable concertina structure 3328 may be formed as a series of ridges and grooves on the surface of the tubes 3350. The concertina structure 3328 may be biased toward a retracted position, and may move to an expanded position when the patient dons the positioning and stabilising structure 3300. Because portions of the tubes 3350 may be substantially inextensible (e.g., non-extendable tube sections 3363), the concertina structures 3328 permit the positioning and stabilising structure 3300 to stretch in order to fit different sized heads. This may allow a single sized tube 3350 to be used with multiple sized heads. For example, the positioning and stabilising structure 3300 may be “one-size-fits-all” as a result of the concertina structure 3328. Alternatively, the tubes 3350 may be manufactured in multiple sizes (e.g., small, medium, large). The patient may select a length that most closely conforms to their head, and the concertina structures 3328 may make small adjustments in order to tailor the fit to the individual patient.

In some forms, the inlet 3332 may be disposed in the middle of the conduit 6320. For example, the tubes 3350 may be symmetric about the inlet 3332 through at least one axis.

The cross-sectional shape of the non-extendable tube sections 3363 of the tubes 3350 may be circular, elliptical, oval, D-shaped or a rounded rectangle, for example as described in U.S. Pat. No. 6,044,844. A cross-sectional shape that presents a flattened surface of tube on the side that faces and contacts the patient's face or other part of the head may be more comfortable to wear than, for example a tube with a circular cross-section.

In some examples of the present technology, the non-extendable tube sections 3363 connects to the plenum chamber 3200 from a low angle. The headgear tubes 3350 may extend inferiorly down the sides of the patient's head and then curve anteriorly and medially to connect to the plenum chamber 3200 in front of the patient's face. The tubes 3350, before connecting to the plenum chamber 3200, may extend to a location at the same vertical position as (or, in some examples, inferior to) the connection with the plenum chamber 3200. That is, the tubes 3350 may project in an at least partially superior direction before connecting with the plenum chamber 3200. A portion of the tubes 3350 may be located inferior to the plenum chamber 3200 and/or the seal forming structure 3100. The tubes 3350 may contact the patient's face below the patient's cheekbones, which may be more comfortable than contact on the patient's cheekbones and may avoid excessively obscuring the patient's peripheral vision.

### 5.3.3.1.3 Conduit Headgear Connection Port

In certain forms of the present technology, the patient interface 3000 may comprise a connection port 3600 located proximal to a superior, lateral or posterior portion of a patient's head. For example, in the form of the present technology illustrated in FIG. 3Z, the connection port 3600 is located on top of the patient's head (e.g. at a superior location with respect to the patient's head). In this example the patient interface 3000 comprises an elbow 3610 forming the connection port 3600. The elbow 3610 may be configured to fluidly connect with a conduit of an air circuit 4170. The elbow 3610 may be configured to swivel with respect to the positioning and stabilising structure 3300 to at least partially decouple the conduit from the positioning and stabilising structure 3300. In some examples the elbow 3610 may be configured to swivel by rotation about a substantially vertical axis and, in some particular examples, by rotation about two or more axes. In some examples the elbow may comprise or be connected to the tubes 3350 by a ball-and-socket joint. The connection portion 3600 may be located in the sagittal plane of the patient's head in use.

Patient interfaces having a connection port that is not positioned anterior to the patient's face may be advantageous as some patients may find a conduit that connects to a patient interface anterior to their face to be unsightly and/or obtrusive. For example, a conduit connecting to a patient interface anterior to the patient's face may be prone to interference with bedclothes or bed linen, particularly if the conduit extends inferiorly from the patient interface in use. Forms of the present technology comprising a patient interface having a connection port positioned superiorly to the patient's head in use may make it easier or more comfortable for a patient to lie or sleep in one or more of the following positions: a side-sleeping position, a supine position (e.g. on their back, facing generally upwards) or in a prone position (e.g. on their front, facing generally downwards). Moreover, connecting a conduit to an anterior portion of a patient interface may exacerbate a problem known as tube drag in which the conduit exerts an undesired force upon the patient interface during movement of the patient's head or the conduit, thereby causing dislodgement away from the face. Tube drag may be less of a problem when force is received at a superior location of the patient's head than anterior to the patient's face proximate to the seal-forming structure (where tube drag forces may be more likely to disrupt the seal).

### 5.3.3.1.4 Headgear Tube Fluid Connections

The two tubes 3350 are fluidly connected at their inferior ends to the plenum chamber 3200. In certain forms of the technology, the connection between the tubes 3350 and the plenum chamber 3200 is achieved by connection of two rigid connectors. The tubes 3350 and plenum chamber 3200 may be configured to enable the patient to easily connect the two components together in a reliable manner. The tubes 3350 and plenum chamber 3200 may be configured to provide tactile and/or audible feedback in the form of a ‘re-assuring click’ or a similar sound, so that the patient may easily know that each tube 3350 has been correctly connected to the plenum chamber 3200. In one form, the tubes 3350 are formed from a silicone or textile material and the inferior end of each of the silicone tubes 3350 is overmolded to a rigid connector made, for example, from polypropylene, polycarbonate, nylon or the like. The rigid connector on each tube 3350 may comprise a female mating feature configured to connect with a male mating feature on the plenum chamber 3200. Alternatively, the rigid connector on each tube 3350 may comprise a male mating feature configured to connect to a female mating feature on the plenum chamber 3200. In other examples the tubes 3350 may each comprise a male or female connector formed from a flexible material, such as silicone or TPE, for example the same material from which the tubes 3350 are formed.

In other examples a compression seal is used to connect each tube 3350 to the plenum chamber 3200. For example, a resiliently flexible (e.g. silicone) tube 3350 without a rigid connector may be configured to be squeezed to reduce its diameter so that it can be compressed into a port in the plenum chamber 3200 and the inherent resilience of the silicone pushes the tube 3350 outwards to seal the tube 3350 in the port in an air-tight manner. Alternatively, in a hard-to-hard type engagement between the tube 3350 and the plenum chamber 3200, each tube 3350 and/or plenum chamber 3200 may comprise a pressure activated seal, for example a peripheral sealing flange. When pressurised gas is supplied through the tubes 3350 the sealing flange may be urged against the join between the tubes and a circumferential surface around a port or connector of the plenum chamber 3200 to form or enhance a seal between the tube 3350 and plenum chamber 3200.

### 5.3.3.2 Headgear Straps

In some forms, the positioning and stabilising structure 3300 may include headgear 3302 with at least one strap which may be worn by the patient in order to assist in properly orienting the seal-forming structure 3100 against the patient's face (e.g., in order to limit or prevent leaks).

As described above, some forms of the headgear 3302 may be constructed from a textile material, which may be comfortable against the patient's skin. The textile may be flexible in order to conform to a variety of facial contours. Although the textile may include rigidisers along a selected length, which may limit bending, flexing, and/or stretching of the headgear 3302.

In certain forms, the headgear 3302 may be at least partially extensible. For example, the headgear 3302 may include elastic, or a similar extensible material. For example, the entire headgear 3302 may be extensible or selected portions may be extensible (or more extensible than surrounding portions). This may allow the headgear 3302 to stretch while under tension, which may assist in providing a sealing force for the seal-forming structure 3100.

Two forms of the headgear, four-point headgear 3302-1 and two-point headgear 3302-2, are discussed in more detail below as illustrative examples.

### 5.3.3.2.1 Four-Point Connection

As shown in FIG. 7E, some forms of the headgear 3302-1 may be a four-point connection headgear. This means that the headgear 3302-1 may connect to four separate places on the plenum chamber 3200, on a frame connected to the plenum chamber 3200, and/or on arms connected to the plenum chamber 3200. The headgear 3302-1 may include four different straps providing a tensile force to help maintain the seal-forming structure 3100 in a sealing position. The positioning and stabilising structure 3300 of FIG. 3A may also be considered a four-point connection headgear.

In some forms, the headgear 3302-1 may include inferior straps 3304-1, which may connect to an inferior portion of the cushion 3050-1. The inferior straps 3304-1 may extend along the patient's cheek toward a posterior region of the patient's head. For example, the inferior straps 3304-1 may overlay the masseter muscle on either side of the patient's face. The inferior straps 3304-1 may therefore contact the patient's head below the patient's ears. The inferior straps 3304-1 may meet at the posterior of the patient's head, and may overlay the occipital bone and/or the trapezius muscle.

The headgear 3302-1 may also include superior straps 3305-1, which may overlay the temporal bones, parietal bone, and/or occipital bone. The superior straps 3305-1 may also connect to the tubes 3350 (e.g., by interfacing with the tabs 3320).

A rear strap 3307-1 may extend between the superior straps 3305-1 and between the inferior straps 3304-1. The inferior and superior straps 3304-1, 3305-1 on a given side (e.g., left or right) may also be connected to the rear strap 3307-1 adjacent to one another. The height of the rear strap 3307-1 may therefore be approximately the combined height of the inferior and superior strap 3304-1, 3305-1. The rear strap 3307-1 may overlay the occipital bone and/or the parietal bone in use. This may allow the rear strap 3307-1 to assist in anchoring the headgear 3302-1 to the patient's head.

In the illustrated example, the headgear 3302-1 may be formed with a substantially X-shape. The inferior and superior straps 3304-1, 3305-1 may be connected to a rear strap 3307-1 using stitching, ultrasonic welding, or any similar process.

In some forms, the inferior straps 3304-1 are connected to a magnetic member 3306-1. For example, each inferior straps 3304-1 may be threaded through a magnetic member 3306-1, so that a length of each inferior strap 3304-1 may be adjusted. The magnetic members 3306-1 may removably connect to the magnets 3370-1 (described below), so that the inferior straps 3304-1 may be disconnected from the plenum chamber 3200, but the length of the inferior straps 3304-1 may not be affected.

In some forms, the superior straps 3305-1 may be connected directly to the tabs 3320 of the tubes 3350. The superior straps 3305-1 may be threaded through the tabs 3320 in order to adjust the length and control the tensile force of each superior strap 3305-1.

In some forms, the headgear 3302-1 may be used only with the nose and mouth cushion 3050-1 (e.g., because the nose-only cushion 3050-1 does not have four connection points). However, the headgear 3302-1 may be used interchangeably with the tubes 3350 and the rigidiser arms 3340.

### 5.3.3.2.2 Two-Point Connection

As shown in FIG. 7F, some forms of the headgear 3302-2 may be a two-point connection headgear. This means that the headgear 3302-2 may connect to two separate places.

In some forms, the headgear 3302-2 may be formed from a continuous piece of material. In other words, the headgear 3302-2 may not be formed from multiple straps connected (e.g., stitched) together. This may be comfortable for a patient as they will not be in contact with any seams or joints connecting different straps. In other forms, the headgear 3302-2 may be formed from multiple straps (e.g., two superior straps, a rear strap, etc.) that are connected together (e.g., with stitching, ultra-sonic welding, etc.).

In certain forms of the present technology, the positioning and stabilising structure 3300 comprises at least one headgear strap acting in addition to the tubes 3350 to position and stabilise the seal-forming structure 3100 at the entrance to the patient's airways. As shown in FIG. 3Z, the patient interface 3000 comprises a strap 3307-2 forming part of the positioning and stabilising structure 3300. The strap 3307-2 may be known as a back strap or a rear headgear strap, for example. The rear strap 3307-2 may overlay the temporal bones, parietal bone, and/or occipital bone. In other examples of the present technology, one or more further straps may be provided. For example, patient interfaces 3000 according to examples of the present technology having a nose-and-mouth cushion may have a second, lower, strap configured to lie against the patient's head proximate the patient's neck and/or against posterior surfaces of the patient's neck.

In the example shown in FIG. 3Z, strap 3310 of the positioning and stabilising structure 3300 is connected between the two tubes 3350 positioned on each side of the patient's head and passing around the back of the patient's head, for example overlying or lying inferior to the occipital bone of the patient's head in use. The strap 3310 connects to each tube above the patient's ears. With reference to FIG. 3Z, the positioning and stabilising structure 3300 comprises a pair of tabs 3320. In use a strap 3310 may be connected between the tabs 3320. The strap 3310 may be sufficiently flexible to pass around the back of the patient's head and lie comfortably against the patient's head, even when under tension in use.

As shown in FIG. 7F, some forms of the headgear 3302-2 may be at least partially bifurcated. For example, a rear strap 3307-2 of the headgear 3302-2 (e.g., configured to contact the posterior portion of the patient's head) may be wider than the surrounding portions of the headgear 3302-2. An intermediate section 3308-2 of the rear strap 3307-2 may include a slit 3309-2. A superior section of the rear strap 3307-2 may therefore be movable relative to the inferior section as a result of the slit 3309-2. This may allow the patient to have a larger strap coverage on the posterior region of their head, which may assist in better anchoring the headgear 3302-2 to the patient's head since there is no inferior strap (e.g., 3304-1).

In some forms, the headgear 3302-2 may be used only with the nasal cushion 3050-2 (e.g., because the nose and mouth cushion 3050-1 does not have four connection points). However, the headgear 3302-2 may be used interchangeably with the tubes 3350 and the rigidiser arms 3340.

### 5.3.3.3 Rigidiser Arm

As shown in FIG. 7D, a rigidiser arm 3340 may be an elongated, rigid member that assists in maintaining the cushion (e.g., the nose and mouth cushion 3050-1 or the nasal cushion 3050-2) in an operating position. The rigidiser arm 3340 may contact a side of the patient's head and provide a force to limit slipping of the seal-forming structure 3100 from the patient's nose and/or mouth.

In some forms, the rigidiser arm 3340 is constructed from a rigid material (e.g., plastic). The rigid material may not permit the rigidiser arm 3340 to stretch. Additionally, the rigidiser arm 3340 may be substantially inflexible and may be unable to bend. The rigidiser arm 3340 may be pre-molded into a desired shape in order to fit a patient's head. For example, the rigidiser arms 3340 may be molded with a curved shape to substantially correspond to the shape of the side of the patient's head (e.g., overlaying the masseter muscle and/or the temporal bone).

In certain forms, the rigidiser arm 3340 may be molded in order to conform to a specific patient's head (e.g., the rigidiser arm 3340 is customized).

In some forms, the rigidiser arm 3340 may be flexible along at least one direction. For example, the rigidiser arm 3340 may be flexible about its width and may be inflexible along its length. In other words, the rigidiser arm 3340 may be bendable about an axis along the width of the rigidiser arm 3340, but may be unable to bend about an axis perpendicular to the rigidiser arm 3340. This may allow an individual patient to adjust the rigidiser arm 3340 in order to better fit their individual head.

In certain forms, the rigidiser arm 3340 may remain in the new position after being bent. This may allow a patient adjust the shape of the rigidiser arm 3340 for their specific head and then the rigidiser arm 3340 will keep the desired shape while in use in order to promote patient comfort.

In some forms, a first end 3342 of the rigidiser arm 3340 may be a free end and a second end 3344 (e.g., opposite of the first end 3342) of the rigidiser arm 3340 may be fixed. The first end 3342 may be curved in order to minimize sharp edges that could cause patient discomfort. The first end 3342 may also overlay the patient's head proximate to the temporal bone, in use. The second end 3344 may be fixed to an arm connection structure 3504.

In some forms, the arm connection structure 3504 may be similar to the conduit connection structure 3500. For example, the arm connection structure 3504 and the conduit connection structure 3500 may have substantially the same shape. This may allow either the conduit connection structure 3500 or the arm connection structure 3504 to fit into the groove (e.g., 3266-1 or 3266-2) and connect to the plenum chamber inlet port 3254. The arm connection structure 3504 may connect to the nose and mouth cushion 3050-1 or the nose-only cushion 3050-2 in substantially the same way as the conduit connection structure 3500 (e.g., via a snap fit, press fit, friction fit, etc.).

In some forms, the arm connection structure 3504 may act as a plug for the plenum chamber inlet port 3254 (e.g., either 3254-1 and/or 3254-2). Unlike the tubes 3350, the rigidiser arm 3340 does not convey pressurized air to the plenum chamber 3200. The rigidised arm 3340 may be used with a “tube down” configuration, where a hose is connected to the vent opening 3402 (e.g., either 3402-1 and/or 3402-2), and conveys air into the plenum chamber 3200 through the vent opening 3402. In this example, air does not need to travel into or out of the plenum chamber inlet openings 3254. Thus, the arm connection structure 3504 may form a seal with the plenum chamber inlet opening 3254 in order to limit airflow into or out of the plenum chamber 3200.

### 5.3.4 Further Examples of Two-Point Connection Headgear

Referring next to FIGS. 8-10, in one form of the technology, which may be particularly suitable for a nose-only mask, the positioning and stabilising structure 3300 comprises side portions or straps 3312, a top portion or strap 3314 and a rear portion or strap 3316. The top portion 3314 may be configured to engage a superior portion of the patient's head in use and may, for example, be configured to overlie the parietal bones of the patient's head in use. The rear portion 3316 may be configured to engage a posterior portion of the patient's head in use and may, for example, be configured to overlie the occipital bone of the patient's head in use, or lie proximate a junction between the occipital bone and the parietal bones.

In examples, a cushion module 3270, which comprises the seal forming structure 3100, is connected to a frame or frame portion 3272 and the side portions or straps 3112 are connected to the frame 3270 by suitable connectors, for example loops 3274. Each side portion 3312 may extend from the frame or frame portion of the patient interface 3000 and a junction of the top portion 3314 and the rear portion 3316. Each side portion 3312 may lie on a path extending between a respective eye and ear of the patient.

In examples, one or more of the straps or portions may comprise a textile. In examples the straps or portions comprise a foam covered by textile.

In each of the examples shown in FIGS. 8-10 the seal-forming structure 3100 may form a seal surrounding the patient's nasal airways and, for example, may be configured to form a seal in use to at least the patient's lip superior, nasal alae and a pronasale portion of the patient's nose.

In examples the side straps or portions 3312 may comprise a rigidiser arm. The rigidiser arm may be an elongate member that provides an increased level of rigidity to the respective side portion 3312 compared to some other portions of the positioning and stabilising structure 3300. In some forms, the rigidiser arm is constructed so as to be rigid or semi-rigid in at least one direction. For example, the rigidiser arm may be constructed from a relatively hard material. Additionally, the rigidiser arm may be constructed in a shape that makes deformation of the rigidiser arm in one or more directions difficult, but the rigidiser arm may be flexible in at least one direction. The rigidiser arm may be pre-moulded in a desired shape in order to fit the patient's head. In some forms the patient may be able to further deform the rigidiser arm to a required shape. In examples the side straps or portions 3312 may comprise a sleeve. The rigidiser arm may be positioned inside the sleeve. In examples the rigidiser arm may be attached to the sleeve, for example with an adhesive and/or heat-activated material.

Examples of techniques for incorporating rigidisers into headgear are disclosed in Australian Patent Application No. 2023902766, the content of which is incorporated herein by reference.

5.3.5 Headgear with Mouth Blocking Portion

Referring next to FIG. 9, in one form of the technology the positioning and stabilising structure 3300 further comprises an inferior strap or portion 3318 comprising a mouth blocking member or portion 6000. In examples, the mouth blocking member 6000 comprises a mouth engaging portion 6010. In one example, the mouth engaging portion 6010 may be made from silicone. In examples, the patient facing surface of the silicone may be slightly “tacky”, which may assist in ensuring the lips do not move apart. In other examples the mouth engaging portion 6010 may be made from an alternative bio-compatible substantially non-porous material. In one example, the mouth engaging portion 6010 may comprise a textile with a suitable substantially non-porous coating. In examples the mouth engaging portion 6010 may be formed integrally with the inferior strap or portion 3318. Alternatively, the mouth engaging portion 6010 may be inset into the inferior strap or portion 3318, or may be connected to a patient facing surface of the inferior strap or portion 3318.

In examples, the inferior strap or portion 3318 extends inferior to the patient's ears and is connected to the rear portion or strap 3316 at the rear of the patient's head.

The mouth engaging portion 6010 may form a seal with the patient's lips, in use, and may thereby reduce or eliminate mouth leak.

The inferior strap or portion 3318 may comprise adjustment means (not shown) for adjusting its length, and thereby adjusting the force with which the mouth engaging portion 6010 engages the patient's lips. Additionally, or alternatively, the inferior strap or portion 3318 may be elastic.

5.3.6 Headgear with Chin Strap

Referring next to FIG. 10, in another form of the technology the positioning and stabilising structure 3300 may comprise a chin strap or chin support portion 6100. The chin strap 6100 may be connected to the side straps or portion 3312, for example substantially level with and anterior of the patient's ears.

The chin strap 6100 may comprise a chin engaging portion 6110 which, in examples, may be wider the than the remainder of the chin strap 6100. In examples, the chin engaging portion 6110 may be shaped to have a substantially concave portion to conform to the patient's chin.

In use, the chin strap 6100 may apply a force to the patient's chin which biases the patient's chin towards a position in which the patient's mouth is closed. By keeping the patient's mouth closed, the chances of the patient experiencing mouth leak are reduced. The chin strap 6100 may comprise adjustment means (not shown) for adjusting the length of the chin strap 6100 and thereby the biasing force on the patient's chin. Additionally, or alternatively, the chin strap 6100 may be elastic.

In another form of the technology, as shown in FIG. 11, the strap 3318 or 6100 may serve as a chin strap or a mouth seal. This may be accomplished by allowing the same strap to move or alternate between the positions shown in FIG. 9 and FIG. 10, whilst making adjustments (e.g., lengthwise via hook and loop fastener and/or one or more buckles, the straps 3312, 3316 including a tab with a slot through which the strap is adjustably connected, etc.) to ensure a good fit.

The strap or straps could be made in modular form such that the chin cup or the nose engaging portion could be swapped out depending on whether the user wishes to cover the mouth or support the chin.

In one form, the strap can remain attached to the positioning and stabilizing structure at the same point of connection, whether in the mouth sealing mode or the chin strap mode.

However, to achieve more ideal force vectors appropriate for the mouth seal or the chin strap, the ends of the strap may be connected to the remaining part of the positioning and stabilizing structure at different points (e.g., exemplary points being shown in FIGS. 9-10). Moreover, the strap can be detached from the positioning and stabilizing structure as desired by the patient.

In another form, the strap can both seal the mouth and serve as a chin support, as shown in FIG. 11. In this example, the strap could be attached to strap 3312 (e.g., rigidized portion thereof), strap 3316 or simultaneously to both straps 3312 and 3316 (e.g., in the positions shown in FIGS. 9 and 10). In alternative, both straps 3318 and 6110 could be used at the same time, or they could be integrated with the ability to be detached from one another (e.g., where they cross) in case the user only desires to use one of them. The two straps could be adjustably connected to one another.

### 5.3.7 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

As shown in FIG. 7N, a vent 3450 may be used with the patient interface 3000. The vent 3450 may have a substantially similar shape to the vent opening 3402-1 (e.g., a substantially circular shape).

The vent 3450 may be used with either the mouth and nose plenum chamber 3200-1 (e.g., illustrated in FIG. 7A) or the nose-only plenum chamber 3200-2 (e.g., illustrated in FIG. 7B).

With continued reference to FIG. 7A, the vent 3450 may include a vent housing 3404, which may be configured to engage with the vent opening 3402. The vent housing 3404 may be constructed from a rigid material or a semi-rigid material. For example, the vent housing 3404 may be constructed from plastic, metal, or any similar material. The vent housing 3404 may add rigidity to the patient interface 3000 (e.g., to limit unwanted bending that may affect the position of the seal-forming structure 3100 on the patient's face).

The vent housing 3404 may include an anterior surface 3408, a posterior surface 3412, and a groove 3416. The anterior surface 3408 faces away from the patient's face in use, and may be positioned outside the pressurized volume of the plenum chamber 3200. The posterior surface 3412 is disposed opposite to the anterior surface 3408. In use, the posterior surface 3412 may face the patient and may be disposed within the pressurized volume of the plenum chamber 3200. The groove 3416 may be formed between the anterior and posterior surfaces 3408, 3412. A portion of the plenum chamber 3200 may be received within the groove 3416 in order to retain the vent 3400 in position.

In some forms, a diffuser 3448 may be used with the vent housing 3404. The diffuser 3448 may assist with limiting the decibel output from any of the patient interface 3000 (or any other patient interface). Specifically, the diffuser 3448 may assist in limiting the decibel level associated with air output from the patient interface 3000 (e.g., exhaled air), although the diffuser 3448 may limit the decibel level of at any point in the patient interface.

In certain forms, the diffuser 3448 may diffuse, and therefore slow, the exhaust gas exiting the plenum chamber 3200 and passing through the vent housing 3404. The diffuser 3448 may assist in avoiding jetting and associated discomfort to the patient and/or bed partner (e.g., noise caused by jetting against a pillow, sheets, bedclothes, etc.).

In some forms, the diffuser may include an anterior surface 3456 that faces away from the patient in use. An outer diameter of the anterior surface 3456 may be less than an inner diameter of the vent housing 3404 proximate to the anterior surface 3408. This may form a gap 3464 through which air may travel.

### 5.3.8 Decoupling Structure(s)

In one form the patient interface 3000 includes at least one decoupling structure, for example, a swivel or a ball and socket.

### 5.3.9 Connection Port

Connection port 3600 allows for connection to the air circuit 4170.

### 5.3.10 Forehead Support

In one form, the patient interface 3000 includes a forehead support 3700.

### 5.3.11 Anti-Asphyxia Valve

In one form, the patient interface 3000 includes an anti-asphyxia valve.

### 5.3.12 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 5.3.13 Modularity

As described above, the cushion, headgear, and sleeves may come in different styles, which may correspond to different uses (e.g., mouth breathing, nasal breathing, etc.). A patient or clinician may select certain combinations of cushions, headgear, and sleeves in order to optimize the effectiveness of the therapy and/or the individual patient's comfort. An example of this sort of modular design is described in PCT/SG2022/050777 filed 28 Oct. 2022, incorporated herein by reference in its entirety.

In some forms, the different styles of cushions, headgear, and sleeves may be used interchangeably with one another in order to form different combinations of patient interfaces. This may be beneficial from a manufacturing prospective because wider variety of patient interfaces may be created using fewer parts. Additionally or alternatively, the various combinations may allow a patient to change styles of patient interface without changing the every component.

Air may be delivered to the patient in one of two main ways. In one example, the patient may receive the flow of pressurized air through headgear tubes 3350 (see e.g., FIG. 3Z). This may be referred to as a “tube up” configuration and may position a connection port at the top of the patient's head. In other example, the patient may receive the flow of pressurized air through a conduit connected to the plenum chamber 3200, for example through the connection port 3600 (see e.g., FIG. 3A). This may be referred to a “tube down” configuration where the airflow conduit is positioned in front of the patient's face. Different patients may be more comfortable with one style of air delivery over the other (e.g., because of the patient's sleep style). Therefore, it may be beneficial to allow a single style of patient interface to be used in either the “tube up” or “tube down” configuration.

The patient interface may be part of a modular assembly with a variety of interchangeable components that may be swapped out by a patient and/or clinician for one or more components for a different style. The following description describes the various combinations that may be created by assembling the different components together.

### 5.3.13.1 Sleeve

In some forms, to allow for modularity, a sleeve may be used with the tubes 3350 and/or the rigidisier arms 3340. The sleeve may at least partially surround the tubes 3350 and/or the rigidiser arms 3340. As shown in FIGS. 7G to 7I, different shapes of sleeves may be used, which may correspond to different types of positioning and stabilising structures 3300. In some forms, the configuration of the sleeve may be customized to fit a particular user's face. For instance, the sleeves may be configured in a relatively more posterior region of the patient's head.

In some forms, the sleeve may be constructed from a comfortable material. For example, the sleeve may be constructed from a textile material, a foam material, or a combination of the two. The comfortable material may contact the patient in use, and may feel soft against the patient's skin in order to improve patient compliance.

The material may also be flexible in order to assist in donning or doffing the sleeve from the tube 3350 or the rigidiser arms 3340. For example, the material may allow the sleeve to bend in order to conform to the shape of the tubes or conduit headgear 3350 or the rigidiser arms 3340, which may change depending on the shape of an individual patient's head.

In some forms, the sleeve may also be at least partially elastic (e.g., the material may allow the sleeve to stretch). The elastic material may help the sleeve stretch in order to fit around the tubes 3350 or the rigidiser arms 3340. The elastic material may then return to an initial position that is snug against the tubes 3350 or the rigidiser arms 3340 in order to limit the sleeve from sliding while in use.

As described in more detail below, some forms of the sleeves may be specific to a rigidising element (e.g., tubes 3350 and/or rigidiser arms 3340). However, the sleeves may assist the rigidising elements in connecting interchangeably with the version or styles of cushions (e.g., the mouth and nose cushion 3050-1, the nose-only cushion 3050-2, etc.).

### 5.3.13.1.1 Conduit Sleeve

As shown in FIG. 7G, one example of a sleeve is a conduit sleeve 3351, which may be usable with the tubes 3350 described above.

As shown in FIG. 7G, the conduit sleeve 3351 may include a curved shape that may be similar to the shape of the tubes 3350 shown in FIG. 7C. The flexible material used to construct the conduit sleeve 3351 may allow the conduit sleeve 3351 to further curve in order to correspond to the shape of the tubes 3350 (e.g., when worn by the patient).

In some forms, the conduit sleeve 3351 may include a first or superior opening 3352. The superior opening 3352 may be disposed at one end of the conduit sleeve 3351. The superior opening 3352 may be an opening to a passage that extends along at least a portion of the conduit sleeve 3351.

As shown in FIG. 7G, some forms of the conduit sleeve 3351 may also include an inferior extension 3354. The inferior extension 3354 may be positioned on an opposite end of the conduit sleeve 3351 from the superior opening 3352. The conduit sleeve 3351 may be customized to fit a particular user's face. For instance, the inferior extension 3354 of the conduit sleeve 3351 may be configured in a relatively more posterior region or anterior region of the patient's head.

Some forms of the inferior extension 3354 may include a rigid or semi-rigid piece (e.g., within the sleeve 3351). The rigid or semi-rigid piece may be constructed from a plastic material, or a similar material. Alternatively, the inferior extension 3354 may be stiffened using a manufacturing process (e.g., stitching rigidised thread, flat knitting, using thicker material).

As shown in FIG. 7G, some forms of the inferior extension 3354 may include a connection member 3356. In the illustrated example, the connection member 3356 may be a magnet, although in other examples, the connection member 3356 may be a different type of connector (e.g., a mechanical fastener, an adhesive, hook and loop material, etc.). The connection member 3356 may also be positioned at an end of the inferior extension 3354, although the connection member 3356 could alternatively be positioned anywhere along the inferior extension 3354.

In some forms, the connection member 3356 (e.g., a magnet) may be removably connected to the magnets 3370-1 of the headgear 3302-1. For example, when the conduit sleeves 3351 are connected to the tubes 3350 (see e.g., FIG. 7J), the magnets 3370-1 connected to the inferior straps 3304-1 may be removably connected to the connection member 3356 in order to provide the tensile force.

### 5.3.13.1.2 Four-Point Arm Sleeve

As shown in FIG. 7H, another example of a sleeve is a four-point arm sleeve 3380, which may be usable with the rigidiser arms 3340 described above.

As shown in FIG. 7H, the four-point arm sleeve 3380 may include a curved shape that may be similar to the shape of the rigidiser arm 3340 shown in FIG. 7D. The flexible material used to construct the four-point arm sleeve 3380 may allow the four-point arm sleeve 3380 to further curve in order to correspond to the shape of the rigidiser arm 3340 (e.g., when worn by the patient and/or went bent by the patient).

As shown in FIG. 7H, some forms of the four-point arm sleeve 3380 may include an inferior extension 3384. The inferior extension 3384 may be positioned at an end of the four-point arm sleeve 3380.

In the illustrated example, the shape and/or structure of the inferior extension 3384 is substantially the same as the shape of the inferior extension 3354. For example, the inferior extension 3384 may be more rigid as compared to the rest of the four-point arm sleeve 3380 (e.g., as a result of rigidising thread or rigid material).

As shown in FIG. 7H, some forms of the inferior extension 3384 may include a connection member 3386. In the illustrated example, the connection member 3386 may be a magnet, although in other examples, the connection member 3386 may be a different type of connector (e.g., a mechanical fastener, an adhesive, hook and loop material, etc.). The connection member 3386 may also be positioned at an end of the inferior extension 3384, although the connection member 3386 could alternatively be positioned anywhere along the inferior extension 3384.

In some forms, the connection member 3386 (e.g., a magnet) may be removably connected to the magnets 3370-1 of the headgear 3302-1. For example, when the four-point arm sleeves 3380 are connected to the rigidiser arm 3340 (see e.g., FIG. 7K), the magnets 3370-1 connected to the inferior straps 3304-1 may be removably connected to the connection member 3386 in order to provide the tensile force.

As shown in FIG. 7H, the four-point arm sleeve 3380 may include a pair of tabs 3394, which may be similar to the tab 3320 on the tubes 3350. When the four-point arm sleeve 3380 is worn by the patient, the tabs 3394 may be positioned in substantially the same place on the patient's head as where the tabs 3320 are positioned when the patient wears the tubes 3350.

### 5.3.13.1.3 Two-Point Arm Sleeve

As shown in FIG. 7I, yet another example of a sleeve is a two-point arm sleeve 3380-1, which may be usable with the rigidiser arms 3340 described above.

In some forms, the two-point arm sleeve 3380-1 may be similar to the four-point arm sleeve 3380 described above. Only some similarities and differences may be described below.

As shown in FIG. 7I, the two-point arm sleeve 3380-1 may include an inferior opening 3388-1 that is positioned at an end of the two-point arm sleeve 3380-1. The inferior opening 3388-1 may form an opening to a passageway through the two-point arm sleeve 3380-1. In the illustrated example, the inferior opening 3388-1 may open into a surface of the conduit sleeve 3380-1.

As shown in FIG. 7I, the two-point arm sleeve 3380-1 may include a pair of tabs 3394-1, which may be similar to the tab 3320 on the tubes 3350. When the two-point arm sleeve 3380-1 is worn by the patient, the tabs 3394-1 may be positioned in substantially the same place on the patient's head as where the tabs 3320 are positioned when the patient wears the tubes 3350.

### 5.3.13.2 Assembled Patient Interfaces

As illustrated in FIGS. 7J to 7M, the various elements described above may be combined into four different patient interfaces. The different patient interfaces may allow patients to use different styles based on their individual comfort. The modularity of the different elements (e.g., the ability to be used in multiple styles of patient interfaces) may simplify manufacturing and/or may allow a patient to more easily switch between styles of patient interfaces.

### 5.3.13.2.1 Nose and Mouth Mask Tube Up Configuration

As illustrated in FIG. 7J, the patient may wear the cushion 3050-1 in a tube-up configuration with the tubes 3350 and the four-point headgear 3302-1. This assembly may form a tube up nose and mouth patient interface 3000-1.

In some forms, a conduit sleeve may be used with the tubes 3350 in order to enable a patient to experience the “tube up” air delivery style with the mouth and nose cushion 3050-1. As is described below, the conduit sleeve provides additional connection locations for connecting the four-point headgear 3302-1. However, other forms of connectors aside from or in addition to the conduit sleeve may be used.

In the illustrated example, the conduit sleeves may be connected to the tubes 3350 of the positioning and stabilising structure 3300. The tubes 3350 (via the conduit connection structure 3500), may be used to connect the tubes 3350 to the cushion 3050-1. The conduit sleeves provide the magnets in order to connect to the magnets 3370-1 (see e.g., FIG. 7E) of the four-point headgear 3302-1. Alternatively, a different connection form may be used.

As illustrated in FIG. 7J, the four-point headgear 3302-1 may connect in four separate locations in order to provide a tensile force that maintains the cushion 3050-1 in a sealing position on the patient's head.

For example, the inferior straps 3304-1 (e.g., via the magnetic members 3306-1) may removably connect to the magnets of the conduit sleeves. In use, each inferior strap 3304-1 may contact the patient's cheek (e.g., overlaying the masseter muscle). The inferior straps 3304-1 may also extend below the patient's ears.

### 5.3.13.2.2 Nose and Mouth Mask Tube Down Configuration

As illustrated in FIG. 7K, the patient may wear the cushion 3050-1 in a tube-down configuration with the rigidiser arms 3340 and the four-point headgear 3302-1. This assembly may form a tube down nose and mouth patient interface 3000-2.

In some forms, a conduit sleeve may be used with the rigidiser arms 3340 in order to enable a patient to experience the “tube down” air delivery style with the mouth and nose cushion 3050-1. As is described below, the conduit sleeve provides additional connection locations for connecting the four-point headgear 3302-1. However, other forms of connectors aside from or in addition to the conduit sleeve may be used.

In the illustrated example, the conduit sleeves may be connected to the rigidiser arms 3340 of the positioning and stabilising structure 3300. The rigidiser arms 3340 (via the conduit connection structure 3504), may be used to connect the rigidiser arms 3340 to the cushion 3050-1. The conduit sleeves provide the magnets in order to connect to the magnets 3370-1 (see e.g., FIG. 7E) of the four-point headgear 3302-1. Alternatively, a different connection form may be used.

As illustrated in FIG. 7K, the four-point headgear 3302-1 may connect in four separate locations in order to provide a tensile force that maintains the cushion 3050-1 in a sealing position on the patient's head.

For example, the inferior straps 3304-1 (e.g., via the magnetic members 3306-1) may removably connect to the magnets of the conduit sleeves. In use, each inferior strap 3304-1 may contact the patient's cheek (e.g., overlaying the masseter muscle). The inferior straps 3304-1 may also extend below the patient's ears.

### 5.3.13.2.3 Nose Mask Tube Up Configuration

As illustrated in FIG. 7L, the patient may wear the cushion 3050-2 in a tube-up configuration with the tubes 3350 and the two-point headgear 3302-2. This assembly may form a tube up nose only patient interface 3000-3

A conduit sleeve may be used with the tubes 3350, and may provide additional comfort to the patient. The sleeve may not add additional connection points to connect the positioning and stabilising structure 3300 on the cushion 3050-2. In the illustrated example, the tubes 3350 of the positioning and stabilising structure 3300 may be connected directly to the cushion 3050-2.

As illustrated in FIG. 7L, the two-point headgear 3302-2 may connect to the tabs 3320 on the tubes 3350 in order to provide a tensile force that maintains the cushion 3050-2 in a sealing position on the patient's head.

### 5.3.13.2.4 Nose Mask Tube Down Configuration

As illustrated in FIG. 7M, the patient may wear the cushion 3050-2 in a tube-up configuration with the rigidiser arms 3340 and the two-point headgear 3302-2. This assembly may form a tube down nose only patient interface 3000-4.

A conduit sleeve may be used with the rigidiser arms 3340, and may provide additional comfort to the patient. The sleeve may not add additional connection points to connect the positioning and stabilising structure 3300 on the cushion 3050-2. In the illustrated example, the rigidiser arms 3340 of the positioning and stabilising structure 3300 may be connected directly to the cushion 3050-2.

As illustrated in FIG. 7M, the two-point headgear 3302-2 may connect to the tabs 3320 on the sleeve in order to provide a tensile force that maintains the cushion 3050-2 in a sealing position on the patient's head.

### 5.3.13.2.5 Modularity of Elements

FIG. 7P illustrates how the different elements can be combined in order to form the four different patient interfaces described above. As illustrated, the different components may be reused for different styles of patient interfaces. This may allow for easier manufacturing and assembly, because a large number of the same components may be produced and used in a variety of styles. The only components not used in multiple styles may be the sleeves. However, the sleeves may be easier to manufacture. FIG. 7O shows a portion of air circuit 4170 that may interface with the patient interface, while FIG. 7N shows a vent 3404 that may interchangeably replace the air circuit shown in FIG. 7O, depending on the style of the patient interface.

### 5.4 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 4 cmH2O, or at least 10 cmH2O, or at least 20 cmH2O.

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, a pressure generator 4140. Electrical components 4200 may be mounted on a single Printed Circuit Board Assembly (PCBA) 4202. In an alternative form, the RPT device 4000 may include more than one PCBA 4202.

### 5.5 Humidifier

### 5.5.1 Humidifier Overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 5.6 Breathing Waveforms

FIG. 6 shows a model typical breath waveform of a person while sleeping. The horizontal axis is time, and the vertical axis is respiratory flow rate. While the parameter values may vary, a typical breath may have the following approximate values: tidal volume Vt 0.5 L, inhalation time Ti 1.6 s, peak inspiratory flow rate Qpeak 0.4 L/s, exhalation time Te 2.4 s, peak expiratory flow rate Qpeak −0.5 L/s. The total duration of the breath, Ttot, is about 4 s. The person typically breathes at a rate of about 15 breaths per minute (BPM), with Ventilation Vent about 7.5 L/min. A typical duty cycle, the ratio of Ti to Ttot, is about 40%.

### 5.7 Glossary

For the purposes of the present technology disclosure, in certain forms of the present technology, one or more of the following definitions may apply. In other forms of the present technology, alternative definitions may apply.

### 5.7.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar˜0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

5.7.1.1 Materials & their Properties

Hardness: Refers to durometer or indentation hardness, which is a material property measured by indentation of an indentor (e.g., as measured in accordance with ASTM D2240).
‘Soft’ materials may include silicone or thermo-plastic elastomer (TPE), and may, e.g. readily deform under finger pressure.
‘Hard’ materials may include polycarbonate, polypropylene, and may not e.g. readily deform under finger pressure.

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 5.7.1.2 Mechanics

### Axes:

a. Neutral axis: An axis in the cross-section of a beam or plate along which there are no longitudinal stresses or strains.
b. Longitudinal axis: An axis extending along the length of a shape. The axis generally passes through a center of the shape.
c. Circumferential axis: An axis oriented perpendicularly with respect to the longitudinal axis. The axis may be specifically present in pipes, tubes, cylinders, or similar shapes with a circular and/or elliptical cross section.

Deformation: The process where the original geometry of a member changes when subjected to forces, e.g. a force in a direction with respect to an axis. The process may include stretching or compressing, bending and, twisting.

Elasticity: The ability of a material to return to its original geometry after deformation.

Floppy structure or component: A structure or component that will change shape, e.g. bend, when caused to support its own weight, within a relatively short period of time such as 1 second.

Resilience: Ability of a material to absorb energy when deformed elastically and to release the energy upon unloading.

Resilient: Will release substantially all of the energy when unloaded. Includes e.g. certain silicones, and thermoplastic elastomers.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Viscous: The ability of a material to resist flow.

Visco-elasticity: The ability of a material to display both elastic and viscous behaviour in deformation.

Yield: The situation when a material can no longer return back to its original geometry after deformation.

### 5.7.1.3 Structural Elements

Compression member: A structural element that resists compression forces.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Membrane: Membrane will be taken to mean a typically thin element that has, preferably, substantially no resistance to bending, but has resistance to being stretched.

Tie (noun): A structure designed to resist tension.

Thin structures:
a. Beams,
   i. A beam may be relatively long in one dimension compared to the other two dimensions such that the smaller dimensions are comparatively thin compared to the long dimension
b. Membranes,
   i. Relatively long in two dimensions, with one thin dimension. Readily deforms in response to bending forces. Resists being stretched, (might also resist compression).
c. Plates & Shells
   i. These may be relatively long in two directions, with one thin dimension. They may have bending, tensile, and/or compressive stiffness.

Thick structures: Solids

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

### 5.7.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Breathing rate: The rate of spontaneous respiration of a patient, usually measured in breaths per minute.

Duty cycle: The ratio of inhalation time, Ti to total breath time, Ttot.

Effort (breathing): The work done by a spontaneously breathing person attempting to breathe.

Expiratory portion of a breathing cycle: The period from the start of expiratory flow to the start of inspiratory flow.

Flow limitation: Flow limitation will be taken to be the state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Types of flow limited inspiratory waveforms:
(i) Flattened: Having a rise followed by a relatively flat portion, followed by a fall.
(ii) M-shaped: Having two local peaks, one at the leading edge, and one at the trailing edge, and a relatively flat portion between the two peaks.
(iii) Chair-shaped: Having a single local peak, the peak being at the leading edge, followed by a relatively flat portion.
(iv) Reverse-chair shaped: Having a relatively flat portion followed by single local peak, the peak being at the trailing edge.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed (obstructed).

Positive End-Expiratory Pressure (PEEP): The pressure above atmosphere in the lungs that exists at the end of expiration.

Peakflow rate (Qpeak): The maximum value of flow rate during the inspiratory portion of the respiratory flow waveform.

Respiratory flow rate, patient airflow rate, respiratory airflow rate (Qr): These terms may be understood to refer to the RPT device's estimate of respiratory flow rate, as opposed to “true respiratory flow rate” or “true respiratory flow rate”, which is the actual respiratory flow rate experienced by the patient, usually expressed in litres per minute.

Tidal volume (Vt): The volume of air inhaled or exhaled during normal breathing, when extra effort is not applied. In principle the inspiratory volume Vi (the volume of air inhaled) is equal to the expiratory volume Ve (the volume of air exhaled), and therefore a single tidal volume Vt may be defined as equal to either quantity. In practice the tidal volume Vt is estimated as some combination, e.g. the mean, of the inspiratory volume Vi and the expiratory volume Ve.

Inhalation Time (Ti): The duration of the inspiratory portion of the respiratory flow rate waveform.

Exhalation Time (Te): The duration of the expiratory portion of the respiratory flow rate waveform.

Total Time (Ttot): The total duration between the start of one inspiratory portion of a respiratory flow rate waveform and the start of the following inspiratory portion of the respiratory flow rate waveform.

Typical recent ventilation: The value of ventilation around which recent values of ventilation Vent over some predetermined timescale tend to cluster, that is, a measure of the central tendency of the recent values of ventilation.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of a rate of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 5.7.3 Ventilation

Adaptive Servo-Ventilator (ASV): A servo-ventilator that has a changeable, rather than fixed target ventilation. The changeable target ventilation may be learned from some characteristic of the patient, for example, a respiratory characteristic of the patient.

Backup rate: A parameter of a ventilator that establishes the minimum breathing rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired interface pressure which the ventilator will attempt to achieve at a given time.

End expiratory pressure (EEP): Desired interface pressure which the ventilator will attempt to achieve at the end of the expiratory portion of the breath. If the pressure waveform template Π(Φ) is zero-valued at the end of expiration, i.e. Π(Φ)=0 when Φ=1, the EEP is equal to the EPAP.

Inspiratory positive airway pressure (IPAP): Maximum desired interface pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts, pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator, or other respiratory therapy device such as an RPT device or portable oxygen concentrator, delivers a volume of breathable gas to a spontaneously breathing patient, it is said to be triggered to do so. Triggering usually takes place at or near the initiation of the respiratory portion of the breathing cycle by the patient's efforts.

### 5.7.4 Anatomy

### 5.7.4.1 Anatomy of the Face

Ala: the external outer wall or “wing” of each nostril (plural: alar)

Alar angle: An angle formed between the ala of each nostril.

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Lip, lower (labrale inferius): The lip extending between the subnasale and the mouth.

Lip, upper (labrale superius): The lip extending between the mouth and the supramenton.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 5.7.4.2 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 5.7.5 Patient Interface

Anti-asphyxia valve (AAV): The component or sub-assembly of a mask system that, by opening to atmosphere in a failsafe manner, reduces the risk of excessive CO2 rebreathing by a patient.

Headgear: Headgear will be taken to mean a form of positioning and stabilising structure designed to hold a device, e.g., a mask, on a head.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’ element per se.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.7.6 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 5.7.6.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 5.7.6.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign. Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical—topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

### 5.7.6.3 Space Curves

Space curves: Unlike a plane curve, a space curve does not necessarily lie in any particular plane. A space curve may be closed, that is, having no endpoints. A space curve may be considered to be a one-dimensional piece of three-dimensional space. An imaginary person walking on a strand of the DNA helix walks along a space curve. A typical human left ear comprises a helix, which is a left-hand helix, see FIG. 3Q. A typical human right ear comprises a helix, which is a right-hand helix, see FIG. 3R. FIG. 3S shows a right-hand helix. The edge of a structure, e.g. the edge of a membrane or impeller, may follow a space curve. In general, a space curve may be described by a curvature and a torsion at each point on the space curve. Torsion is a measure of how the curve turns out of a plane. Torsion has a sign and a magnitude. The torsion at a point on a space curve may be characterised with reference to the tangent, normal and binormal vectors at that point.

Tangent unit vector (or unit tangent vector): For each point on a curve, a vector at the point specifies a direction from that point, as well as a magnitude. A tangent unit vector is a unit vector pointing in the same direction as the curve at that point. If an imaginary person were flying along the curve and fell off her vehicle at a particular point, the direction of the tangent vector is the direction she would be travelling.

Unit normal vector: As the imaginary person moves along the curve, this tangent vector itself changes. The unit vector pointing in the same direction that the tangent vector is changing is called the unit principal normal vector. It is perpendicular to the tangent vector.

Binormal unit vector: The binormal unit vector is perpendicular to both the tangent vector and the principal normal vector. Its direction may be determined by a right-hand rule (see e.g. FIG. 3P), or alternatively by a left-hand rule (FIG. 3O).

Osculating plane: The plane containing the unit tangent vector and the unit principal normal vector. See FIGS. 30 and 3P.

Torsion of a space curve: The torsion at a point of a space curve is the magnitude of the rate of change of the binormal unit vector at that point. It measures how much the curve deviates from the osculating plane. A space curve which lies in a plane has zero torsion. A space curve which deviates a relatively small amount from the osculating plane will have a relatively small magnitude of torsion (e.g. a gently sloping helical path). A space curve which deviates a relatively large amount from the osculating plane will have a relatively large magnitude of torsion (e.g. a steeply sloping helical path). With reference to FIG. 3S, since T2>T1, the magnitude of the torsion near the top coils of the helix of FIG. 3S is greater than the magnitude of the torsion of the bottom coils of the helix of FIG. 3S

With reference to the right-hand rule of FIG. 3P, a space curve turning towards the direction of the right-hand binormal may be considered as having a right-hand positive torsion (e.g. a right-hand helix as shown in FIG. 3S). A space curve turning away from the direction of the right-hand binormal may be considered as having a right-hand negative torsion (e.g. a left-hand helix).

Equivalently, and with reference to a left-hand rule (see FIG. 3O), a space curve turning towards the direction of the left-hand binormal may be considered as having a left-hand positive torsion (e.g. a left-hand helix). Hence left-hand positive is equivalent to right-hand negative. See FIG. 3T.

### 5.7.6.4 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 5.8 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means +/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1. A positioning and stabilising structure for a patient interface, the positioning and stabilising structure comprising a pair of side straps, a top strap and a rear strap, the positioning and stabilising structure further comprising an inferior strap, wherein the inferior strap comprises a mouth blocking member or portion configured to engage and seal against the patient's mouth, to thereby reduce or eliminate mouth leak.

2. The positioning and stabilising structure for a patient interface of claim 1, wherein the rear portion is configured to engage a posterior portion of the patient's head in use and is configured to overlie the occipital bone of the patient's head in use, or lie proximate a junction between the occipital bone and the parietal bones.

3. The positioning and stabilising structure for a patient interface of claim 2, wherein the top portion is configured to engage a superior portion of the patient's head in use and is configured to overlie the parietal bones of the patient's head in use.

4. The positioning and stabilising structure for a patient interface of claim 1, wherein the mouth blocking member or portion comprises a mouth engaging portion. made from silicone.

5. The positioning and stabilising structure for a patient interface of claim 4, wherein a patient facing surface of the silicone is “tacky” to assist in ensuring the lips do not move apart.

6. The positioning and stabilising structure for a patient interface of claim 1, wherein the mouth blocking member or portion comprises a mouth engaging portion made from a bio-compatible and substantially non-porous material.

7. The positioning and stabilising structure for a patient interface of claim 6, wherein the mouth engaging portion comprises a textile with a substantially non-porous coating.

8. The positioning and stabilising structure for a patient interface of claim 6, wherein the mouth engaging portion is formed integrally with the inferior strap.

9. The positioning and stabilising structure for a patient interface of claim 6, wherein the mouth engaging portion is inset into the inferior strap, or may be connected to a patient facing surface of the inferior strap or portion.

10. The positioning and stabilising structure for a patient interface of claim 1, wherein the inferior strap is configured to extend inferior to the patient's ears and is connected to the rear strap at the rear of the patient's head.

11. A patient interface for treating a patient with a respiratory disorder, comprising:
a seal forming portion configured to form a seal with the patient's nasal airways, the seal being configured to form a seal in use with at least the patient's lip superior, nasal alae and a pronasale portion of the patient's nose; and
the positioning and stabilising structure of claim 1.

12. A positioning and stabilising structure for a patient interface, the positioning and stabilising structure comprising a pair of side straps, a top strap and a rear strap, the positioning and stabilising structure further comprising a chin strap comprising a chin engaging portion, the chin strap configured to bias a patient's chin towards a closed mouth position, to thereby reduce or eliminate mouth leak.

13. The positioning and stabilising structure for a patient interface of claim 12, wherein the rear portion is configured to engage a posterior portion of the patient's head in use and is configured to overlie the occipital bone of the patient's head in use, or lie proximate a junction between the occipital bone and the parietal bones.

14. The positioning and stabilising structure for a patient interface of claim 13, wherein the top portion is configured to engage a superior portion of the patient's head in use and is configured to overlie the parietal bones of the patient's head in use.

15. The positioning and stabilising structure for a patient interface of claim 12, wherein the chin strap is connected to the side straps, and is configured to be positioned substantially level with and anterior of the patient's ears.

16. The positioning and stabilising structure for a patient interface of claim 12, wherein the chin engaging portion is wider the than a remainder of the chin strap.

17. The positioning and stabilising structure for a patient interface of claim 12, wherein the chin engaging portion is shaped to have a substantially concave portion to conform to the patient's chin.

18. The positioning and stabilising structure for a patient interface of claim 12, wherein, in use, the chin strap is configured to apply a force to the patient's chin which biases the patient's chin towards a position in which the patient's mouth is closed.

19. The positioning and stabilising structure for a patient interface of claim 12, wherein the chin strap is length adjustable and/or elastic.

20. A patient interface for treating a patient with a respiratory disorder, comprising:
a seal forming portion configured to form a seal with the patient's nasal airways, the seal being configured to form a seal in use with at least the patient's lip superior, nasal alae and a pronasale portion of the patient's nose; and
the positioning and stabilising structure of claim 12.

21. A positioning and stabilising structure for a patient interface, the positioning and stabilising structure comprising a pair of side straps, a top strap and a rear strap, the positioning and stabilising structure further comprising at least one strap comprising a chin engaging portion configured to bias a patient's chin towards a closed mouth position, to thereby reduce or eliminate mouth leak and/or a mouth blocking member or portion configured to engage and seal against the patient's mouth, to thereby reduce or eliminate mouth leak.

22. The positioning and stabilising structure for a patient interface of claim 21, wherein the rear portion is configured to engage a posterior portion of the patient's head in use and is configured to overlie the occipital bone of the patient's head in use, or lie proximate a junction between the occipital bone and the parietal bones, and the top portion is configured to engage a superior portion of the patient's head in use and is configured to overlie the parietal bones of the patient's head in use.

23. The positioning and stabilising structure for a patient interface of claim 21, wherein the strap is configured to move between a mouth sealing position and a chin supporting position.

24. The positioning and stabilising structure for a patient interface of claim 21, wherein the strap remains attached to the side straps or the rear strap at the same point of connection, whether in a mouth sealing mode or a chin strap mode.

25. The positioning and stabilising structure for a patient interface of claim 21, wherein the strap is configured to both seal the mouth and serve as a chin support.

26. The positioning and stabilising structure for a patient interface of claim 21, wherein the strap is attached to the side straps, the rear strap, or simultaneously to both the side and rear straps.

27. A patient interface for treating a patient with a respiratory disorder, comprising:
a seal forming portion configured to form a seal with the patient's nasal airways, the seal being configured to form a seal in use with at least the patient's lip superior, nasal alae and a pronasale portion of the patient's nose; and
the positioning and stabilising structure of claim 21.
